SEQUENCE LISTING (25 sequences)

SEQ ID NO: 1 - DNA, 1473 bp, synthetic construct
  source 1..1473: mol_type = other DNA
tctagaatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgac

SEQ ID NO: 2 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaactacgactccagcaccacgaccaccaacaccagcgcccaccattgcaagccagccactttcactgcgcccagaagcgtgtagaccagctgctggaggagcagtgcatacgagagggctggacttcgcctgtgatatctacatctgggcgcccttggccgggacttgtggggtccttctcctgtcactggttatcaccctttactgccaacagggcaggaagaaactcctgtatatagtcaaacaaccgtttctgaagccagcgcaaacggtccaagaggaagatgcctgcagctgccggtttccagaagaagaggaaggcgagtgtgaactaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgaggaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 3 - DNA, 1608 bp, synthetic construct
  source 1..1608: mol_type = other DNA
atgtgtcacacactgaagtggggaacaccattacccaagctctttcagctcttggtgctggttggtctttttgacttctgttcaggcatcgttcaggtgaccaaaacagtgaaagaaatagcagtgctatcctgtgattacaacatatccactgaagaactgactagagtccgaatatactggcaaaaggataatgaaatggtgctggctgtcatgtctggaaaagtgaaggtgtggcccaagtatgagaaccgcaccttcactgatgtcaccaataacctctgcattgtgatcctggctctgcgcctgtcagacaatggcacctacacctgtgttgttcagaagcgggagagagggtcttataagctggagcacctgacttcggtgaagttaatggtcaaagctgactttcctgtgcctagtattactgcccttggaaatccatctcctaacatcaaaaggataaggtgctcaacctctggaggttttccagagcctcacctctcctggttggaaaatggagaagaattaaatgctaccaacacgatgctttcccaagatcctgaaactgagctctacatgattagcagtgaactggatttcaatgtgacaggcaaccacagcttcatgtgtcttgtcaagtatggaggcttaacagtgtcacagaccttcaactggcaaaaatcagctaaacgagaaacctcttctgctaatcagttcctgctcttcaccgttattatatcagcctcagcatgtgggattgcaataatactaatataccggctctgcagatctgctgctccaagacagagaaggaggaggaatatggagagcatggaaatggaaaggatctccccaatctacccaggatctgtggaaggaccaggcaccgtcggaagcggagctactaacttcagcctgctgaagcaggctggagacgtggaagagaaccctggacctagatccatggccacaaccatgtcgcgcggcctccagcttctgctcctgagctgcgcctacagcctggctcccgcggaacaaaaacttattagcgaagaagatcttgtgctggaggtgaaagtggtctgttcggaggctgtggacttgccctgccccgtcccctgggacccgcaggtccgctacacggtgtcctgggccaagctgtccggcggccgtgcagagagcctggaggtgcccggggaagagctgcggtccgctctccagaatggctcttcgggggcccccagtgggaggctgtattctctgaagatccagaacactaccagctgcaactcgggggcctaccgctgcactctggaggacccggagacccagagaaacctcagcggcaccgtgatcttgaaagtgacaggatgctctaagggacgcaaagaagatacttttaagaaatacagagctgagattgtcctgctgttggctctggtcactttctacttaacactcatcattttcacctgcaagtttgcacgacagcagagtatttttccagatttttctaaaccggtcgtagaacatgcttttctccccgtcacctccccaaataagcccttggagccagtgactcttcacaagacagagctggtataa

SEQ ID NO: 4 - DNA, 2007 bp, synthetic construct
  source 1..2007: mol_type = other DNA
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacggtcggaagcggagctactaacttcagcctgctgaagcaggctggagacgtggaagagaaccctggacctagatccatggccacaaccatgagctccagctccgacgtcgaccaggaccccgaggcccagaggcctcccgcgccccgaggccgagcttgcagccccttgccctgggccttgagcgccgcgctgctagtgttcgcagccgccagcgccgtctgcctggtgcgctcctggaccgcgcccggggcccccgccgcgcccggtcccgcgcccagcccgcgactgccagagggtccggagttgacgtccgacgccctggccggcttcccagactatccacagggcatgttccagcaggatgttttcgcgcagctggtggtggcggagggtgaaaagctgaccgaagggcccctgcactggtgcagcgagccagggatgaaaggtgtgactctgacgccaggtgtgagatatgacagggacacacatgagctggtggtggtcaaggctggaatctactatgtgttcttgcacttggcactgaagcgcgtggtggccgtcaacagcaacagcaacagcaacatcagctccggctccgtctccgttgccctgcacctgcagccttcccacgctgaggccaccgccttggccttgaccttggacctgccacccccatcctcagggaactcagccgctggtttccggggtggcctgctgcacctgggtgctggacagcgcctgggtgtccacctgcaccctgaactccgcaagcccctcacctggcagctcttggacaccacggccttgggcctcttccgtgtggccaccagagcccctgctgtactcccttcacaccagctctcacaccatcatcaccatcactaa

SEQ ID NO: 5 - DNA, 1053 bp, synthetic construct
  source 1..1053: mol_type = other DNA
atgcgcacccccgcccagttcctgggcatcctgctgctgtggttccccggcatcaagtgctacccgtatgatgttccggattacgctgaaacaactgtgacccagtctccagcatccctgtccatggctataggagaacaagtcaccatcagatgcataaccagcactgatattgatgatgatatgaactggtaccagcagaggccagggaaacctcctaagctccttatttcagaaggcaatactcttcgtcctggagtcccatcccgattctccagcagtggctatggtacagattttgtttttacaattgaaaacatgctctcagaggatgttgcagattactactgtttacaaagtgataacttgccgctcacgttcggtgctgggaccaagctggagctgaaaggtggaggcggttcaggcggaggtggctctggcggtggcggatcggaagtgaagctggtggagtctgggggaggcttagtgaagcctggagggtccctgaaactctcctgtgcagcctctggattcactttcagtagctataccatgtcttgggttcgccagactccggagaagaggctggagtgggtcgcatacattagtagtggtggtagtaattcctactattcagacagtgtgaagggccgattcaccatctccagagacaatgccaggaacaccctgtacctgcaaatgagcagtctgaggtctgaggacacggccatgtattactgtgcaagacggggggactatatggactactggggtcaaggaacctcagtcaccgtctcctcagccaagaccaccgcccccagcgtgtaccccctggcccccgtgagcagcggcagcggcagcagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttaa

SEQ ID NO: 6 - DNA, 780 bp, synthetic construct
  source 1..780: mol_type = other DNA
atgagctccagctccgacgtcgaccaggaccccgaggcccagaggcctcccgcgccccgaggccgagcttgcagccccttgccctgggccttgagcgccgcgctgctagtgttcgcagccgccagcgccgtctgcctggtgcgctcctggaccgcgcccggggcccccgccgcgcccggtcccgcgcccagcccgcgactgccagagggtccggagttgacgtccgacgccctggccggcttcccagactatccacagggcatgttccagcaggatgttttcgcgcagctggtggtggcggagggtgaaaagctgaccgaagggcccctgcactggtgcagcgagccagggatgaaaggtgtgactctgacgccaggtgtgagatatgacagggacacacatgagctggtggtggtcaaggctggaatctactatgtgttcttgcacttggcactgaagcgcgtggtggccgtcaacagcaacagcaacagcaacatcagctccggctccgtctccgttgccctgcacctgcagccttcccacgctgaggccaccgccttggccttgaccttggacctgccacccccatcctcagggaactcagccgctggtttccggggtggcctgctgcacctgggtgctggacagcgcctgggtgtccacctgcaccctgaactccgcaagcccctcacctggcagctcttggacaccacggccttgggcctcttccgtgtggccaccagagcccctgctgtactcccttcacaccagctctcatga

SEQ ID NO: 7 - DNA, 543 bp, synthetic construct
  source 1..543: mol_type = other DNA
atggtgctggaggtgaaagtggtctgttcggaggctgtggacttgccctgccccgtcccctgggacccgcaggtccgctacacggtgtcctgggccaagctgtccggcggccgtgcagagagcctggaggtgcccggggaagagctgcggtccgctctccagaatggctcttcgggggcccccagtgggaggctgtattctctgaagatccagaacactaccagctgcaactcgggggcctaccgctgcactctggaggacccggagacccagagaaacctcagcggcaccgtgatcttgaaagtgacaggatgctctaagggacgcaaagaagatacttttaagaaatacagagctgagattgtcctgctgttggctctggtcactttctacttaacactcatcattttcacctgcaagtttgcacgacagcagagtatttttccagatttttctaaaccggtcgtagaacatgcttttctccccgtcacctccccaaataagcccttggagccagtgactcttcacaagacagagctggta

SEQ ID NO: 8 - DNA, 891 bp, synthetic construct
  source 1..891: mol_type = other DNA
atgtgtcacacactgaagtggggaacaccattacccaagctctttcagctcttggtgctggttggtctttttgacttctgttcaggcatcgttcaggtgaccaaaacagtgaaagaaatagcagtgctatcctgtgattacaacatatccactgaagaactgactagagtccgaatatactggcaaaaggataatgaaatggtgctggctgtcatgtctggaaaagtgaaggtgtggcccaagtatgagaaccgcaccttcactgatgtcaccaataacctctgcattgtgatcctggctctgcgcctgtcagacaatggcacctacacctgtgttgttcagaagcgggagagagggtcttataagctggagcacctgacttcggtgaagttaatggtcaaagctgactttcctgtgcctagtattactgcccttggaaatccatctcctaacatcaaaaggataaggtgctcaacctctggaggttttccagagcctcacctctcctggttggaaaatggagaagaattaaatgctaccaacacgatgctttcccaagatcctgaaactgagctctacatgattagcagtgaactggatttcaatgtgacaggcaaccacagcttcatgtgtcttgtcaagtatggaggcttaacagtgtcacagaccttcaactggcaaaaatcagctaaacgagaaacctcttctgctaatcagttcctgctcttcaccgttattatatcagcctcagcatgtgggattgcaataatactaatataccggctctgcagatctgctgctccaagacagagaaggaggaggaatatggagagcatggaaatggaaaggatctccccaatctacccaggatctgtggaaggaccaggcacc

SEQ ID NO: 9 - DNA, 1125 bp, synthetic construct
  source 1..1125: mol_type = other DNA
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtga

SEQ ID NO: 10 - DNA, 789 bp, synthetic construct
  source 1..789: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 11 - DNA, 207 bp, synthetic construct
  source 1..207: mol_type = other DNA
actacgactccagcaccacgaccaccaacaccagcgcccaccattgcaagccagccactttcactgcgcccagaagcgtgtagaccagctgctggaggagcagtgcatacgagagggctggacttcgcctgtgatatctacatctgggcgcccttggccgggacttgtggggtccttctcctgtcactggttatcaccctttactgc

SEQ ID NO: 12 - DNA, 126 bp, synthetic construct
  source 1..126: mol_type = other DNA
caacagggcaggaagaaactcctgtatatagtcaaacaaccgtttctgaagccagcgcaaacggtccaagaggaagatgcctgcagctgccggtttccagaagaagaggaaggcgagtgtgaacta

SEQ ID NO: 13 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgaggaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCQQGRKKLLYIVKQPFLKPAQTVQEEDACSCRFPEEEEGECELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 263 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 16 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 17 - protein, 209 aa, synthetic construct
MSRGLQLLLLSCAYSLAPAEQKLISEEDLVLEVKVVCSEAVDLPCPVPWDPQVRYTVSWAKLSGGRAESLEVPGEELRSALQNGSSGAPSGRLYSLKIQNTTSCNSGAYRCTLEDPETQRNLSGTVILKVTGCSKGRKEDTFKKYRAEIVLLLALVTFYLTLIIFTCKFARQQSIFPDFSKPVVEHAFLPVTSPNKPLEPVTLHKTELV

SEQ ID NO: 18 - protein, 297 aa, synthetic construct
MCHTLKWGTPLPKLFQLLVLVGLFDFCSGIVQVTKTVKEIAVLSCDYNISTEELTRVRIYWQKDNEMVLAVMSGKVKVWPKYENRTFTDVTNNLCIVILALRLSDNGTYTCVVQKRERGSYKLEHLTSVKLMVKADFPVPSITALGNPSPNIKRIRCSTSGGFPEPHLSWLENGEELNATNTMLSQDPETELYMISSELDFNVTGNHSFMCLVKYGGLTVSQTFNWQKSAKRETSSANQFLLFTVIISASACGIAIILIYRLCRSAAPRQRRRRNMESMEMERISPIYPGSVEGPGT

SEQ ID NO: 19 - protein, 374 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 20 - protein, 535 aa, synthetic construct
MCHTLKWGTPLPKLFQLLVLVGLFDFCSGIVQVTKTVKEIAVLSCDYNISTEELTRVRIYWQKDNEMVLAVMSGKVKVWPKYENRTFTDVTNNLCIVILALRLSDNGTYTCVVQKRERGSYKLEHLTSVKLMVKADFPVPSITALGNPSPNIKRIRCSTSGGFPEPHLSWLENGEELNATNTMLSQDPETELYMISSELDFNVTGNHSFMCLVKYGGLTVSQTFNWQKSAKRETSSANQFLLFTVIISASACGIAIILIYRLCRSAAPRQRRRRNMESMEMERISPIYPGSVEGPGTVGSGATNFSLLKQAGDVEENPGPRSMATTMSRGLQLLLLSCAYSLAPAEQKLISEEDLVLEVKVVCSEAVDLPCPVPWDPQVRYTVSWAKLSGGRAESLEVPGEELRSALQNGSSGAPSGRLYSLKIQNTTSCNSGAYRCTLEDPETQRNLSGTVILKVTGCSKGRKEDTFKKYRAEIVLLLALVTFYLTLIIFTCKFARQQSIFPDFSKPVVEHAFLPVTSPNKPLEPVTLHKTELV

SEQ ID NO: 21 - protein, 350 aa, synthetic construct
MRTPAQFLGILLLWFPGIKCYPYDVPDYAETTVTQSPASLSMAIGEQVTIRCITSTDIDDDMNWYQQRPGKPPKLLISEGNTLRPGVPSRFSSSGYGTDFVFTIENMLSEDVADYYCLQSDNLPLTFGAGTKLELKGGGGSGGGGSGGGGSEVKLVESGGGLVKPGGSLKLSCAASGFTFSSYTMSWVRQTPEKRLEWVAYISSGGSNSYYSDSVKGRFTISRDNARNTLYLQMSSLRSEDTAMYYCARRGDYMDYWGQGTSVTVSSAKTTAPSVYPLAPVSSGSGSSKNTLVLFGAGFGAVITVVVIVVIIKCFCKHRSCFRRNEASRETNNSLTFGPEEALAEQTVFL

SEQ ID NO: 22 - protein, 668 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGATVGSGATNFSLLKQAGDVEENPGPRSMATTMSSSSDVDQDPEAQRPPAPRGRACSPLPWALSAALLVFAAASAVCLVRSWTAPGAPAAPGPAPSPRLPEGPELTSDALAGFPDYPQGMFQQDVFAQLVVAEGEKLTEGPLHWCSEPGMKGVTLTPGVRYDRDTHELVVVKAGIYYVFLHLALKRVVAVNSNSNSNISSGSVSVALHLQPSHAEATALALTLDLPPPSSGNSAAGFRGGLLHLGAGQRLGVHLHPELRKPLTWQLLDTTALGLFRVATRAPAVLPSHQLSHHHHHH

SEQ ID NO: 23 - protein, 259 aa, synthetic construct
MSSSSDVDQDPEAQRPPAPRGRACSPLPWALSAALLVFAAASAVCLVRSWTAPGAPAAPGPAPSPRLPEGPELTSDALAGFPDYPQGMFQQDVFAQLVVAEGEKLTEGPLHWCSEPGMKGVTLTPGVRYDRDTHELVVVKAGIYYVFLHLALKRVVAVNSNSNSNISSGSVSVALHLQPSHAEATALALTLDLPPPSSGNSAAGFRGGLLHLGAGQRLGVHLHPELRKPLTWQLLDTTALGLFRVATRAPAVLPSHQLS

SEQ ID NO: 24 - protein, 42 aa, synthetic construct
QQGRKKLLYIVKQPFLKPAQTVQEEDACSCRFPEEEEGECEL

SEQ ID NO: 25 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0025501 A1
(43) Pub. Date: Jan. 23, 2025

(54) GENETICALLY MODIFIED UNGULATE CELLS AND THEIR USES IN CANCER THERAPY

(71) Applicants: IMANIS LIFE SCIENCES, LLC, Rochester, MN (US); MAYO FOUNDATION FOR MEDICAL EDUCATION AND RESEARCH, Rochester, MN (US); FIOS THERAPEUTICS LLC, Rochester, MN (US)

(72) Inventors: Stephen J. Russell, Rochester, MN (US); Karina Krotova, Austin, MN (US); Christopher G.A. McGregor, Rochester, MN (US)

(73) Assignees: IMANIS LIFE SCIENCES, LLC, Rochester, MN (US); MAYO FOUNDATION FOR MEDICAL EDUCATION AND RESEARCH, Rochester, MN (US); FIOS THERAPEUTICS LLC, Rochester, MN (US)

(21) Appl. No.: 18/709,445

(22) PCT Filed: Nov. 14, 2022
(86) PCT No.: PCT/US2022/079820

§ 371 Date: May 10, 2024

Related U.S. Application Data
(60) Provisional application No. 63/278,590, filed on Nov. 12, 2021.

(51) Int. Cl. A61K 35/17; A61K 35/766; A61K 39/00; A61P 35/00; C07K 14/725; C07K 16/28; C12N 5/0783; C12N 15/86

(52) U.S. Cl. CPC A61K 35/17; A61K 35/766; A61K 39/4611; A61K 39/4631; A61K 39/464412; A61P 35/00; C07K 14/7051; C07K 16/2803; C12N 5/0636; C12N 15/86; A61K 2239/13; A61K 2239/22; C07K 2317/622; C12N 2510/00; C12N 2740/15043; C12N 2760/20232

(57) ABSTRACT

Provided herein are genetically modified ungulate cells expressing a chimeric antigen receptor. Also provided herein are transgenic ungulate cells that can be used as cell carriers for the delivery of oncolytic viruses in cancer therapy. Also provided are transgenic ungulates comprising these cells, methods of making these cells, and methods of treating cancer by administering these cells to a subject in need thereof. Also provided are transgenic ungulate cells that express a chimeric antigen receptor and uses thereof.

### PRIOR RELATED APPLICATION

This application claims the benefit of and priority to U.S. Provisional Application No. 63/278,590 filed on Nov. 12, 2021, which is hereby incorporated by reference in its entirety.

### BACKGROUND

CAR T cell immunotherapy is one of the most encouraging developments for the treatment of hematological malignancies. However, biological and manufacturing hurdles can hamper its use, particularly when substantial numbers of cells are needed to treat large patient groups.

Oncolytic virotherapy has also emerged as a promising treatment for cancer. For example, the Food and Drug Administration (FDA) has approved a modified herpes simplex virus (T-VEC or Imlygic®) for certain subsets of patients with melanoma. However, oncolytic virotherapy has challenges, such as penetration into tumors, anti-viral immune responses, off-target infection, and adverse conditions in the tumor microenvironment that limit its usefulness in a wide range of cancers.

### SUMMARY

Provided herein is a genetically modified ungulate cell or a population thereof. The modified ungulate cells comprise a nucleic acid encoding a chimeric antigen receptor (CAR). By way of example, the ungulate cell can be selected from the group consisting of a porcine cell, a bovine cell, and an ovine cell. Optionally, the CAR comprises a human antigen binding domain operably linked to one or more ungulate intracellular signaling domains, such as one or more porcine signaling domains.

Further provided is a pharmaceutical composition comprising (a) any genetically modified ungulate cell provided herein or a population thereof and (b) a pharmaceutically acceptable carrier.

Also provided is a method for treating cancer in a subject comprising administering to the subject (a) a genetically modified ungulate cell provided herein; (b) a population of genetically modified ungulate cells provided herein: or (c) a pharmaceutical composition provided herein. The cancer, for example, can be a solid tumor or a hematological cancer. The cancer can comprise a CD19-expressing cancer. Some methods further comprise administering a second anticancer therapy to the subject. The anticancer therapy can be chemotherapy, immunotherapy, hormone therapy, cytokine therapy, radiotherapy, cryotherapy, or surgical therapy.

Further provided is a method for activating ungulate T cells comprising (a) obtaining a population of ungulate T cells and (b) contacting the population of ungulate T cells with a population of artificial antigen presenting cells, wherein the antigen presenting cells express ungulate CD80, ungulate CD83, and an anti-ungulate CD3 antibody. The population of ungulate T cells is optionally a population of porcine T cells. The artificial antigen presenting cells (e.g., artificial dendritic cells) optionally express a porcine CD80 and a porcine CD83, and the anti-CD3 antibody is an anti-porcine CD3 antibody. In some methods, the anti-CD3 antibody is a scFv anti-CD3 antibody.

The population of ungulate cells (e.g., a population of porcine T cells) can be plated on a retronectin-coated dish in culture media comprising human IL-2 and ungulate IL-21 (e.g., porcine IL-21), prior to contacting the population of ungulate T cells with the artificial antigen presenting cells. The contacting step optionally comprises stimulating the population of porcine T cells with the artificial antigen presenting cells at a ratio of 5:1 (porcine T cells: artificial antigen presenting cells).

Also provided is a method for making a population of genetically modified ungulate T cells comprising a chimeric antigen receptor (CAR) by (a) obtaining a population of activated ungulate T cells produced by any of the cell activation methods provided herein and (b) introducing a nucleic acid encoding the CAR into the population of ungulate T cells. Introducing the nucleic acid optionally comprises transducing the population of activated ungulate T cells with a lentiviral vector comprising a nucleic acid construct encoding the CAR. The activated population of ungulate T cells is optionally a population of porcine T cells. The population of porcine T cells can be plated on retronectin-coated plates. In some methods, the population of plated porcine T cells is transduced in the presence of polybrene. The population of plated transduced porcine T cells is optionally expanded by culturing the population in culture media comprising human IL-2 and porcine IL-21.

The lentiviral vector optionally comprises a nucleic acid encoding the CAR. The CAR can comprise a human antigen binding domain operably linked to one or more ungulate intracellular signaling domains. By way of example, the CAR can be an anti-CD19 CAR, an anti-BCMA CAR, an anti-HER-2 CAR, an anti-EGFR CAR, or an anti-PSA CAR. In some methods, the CAR is a single chain variable fragment (scFv). In some examples. The anti-CD19 CAR, is an anti-human CD19 CAR comprising SEQ ID NO: 15 or a sequence having at least 80%, 85%, 90%, or 95% sequence identity with SEQ ID NO: 15. The lentiviral vector optionally comprises a construct comprising (a) a nucleic acid sequence encoding an anti-CD19 scFv, (b) a nucleic acid encoding a CD8α hinge, (c) a CD8 transmembrane domain, (c) a porcine, cytoplasmic 41BB signaling domain, and (d) a cytoplasmic porcine CD3 zeta signaling domain, wherein the construct is operably linked to a promoter.

Exemplary constructs include constructs comprising the nucleic acid sequence of SEQ ID NO: 1 or SEQ ID NO: 2 or a nucleic acid sequence having at least 95% identity to SEQ ID NO: 1 or SEQ ID NO: 2. The promoter is optionally an EF1-α promoter. An exemplary amino acid sequence comprising an anti-CD19 scFv, a CD8α hinge, a CD8 transmembrane domain, a porcine, cytoplasmic 41BB signaling domain, and a cytoplasmic porcine CD3 zeta signaling domain is set forth herein as SEQ ID NO: 14. In some examples, the anti-CD19 CAR, is an anti-human CD19 CAR comprising SEQ ID NO: 15 or a sequence having at least 80%, 85%, 90%, or 95% sequence identity with SEQ ID NO: 15. In some examples, the amino acid sequence comprising a CD8 hinge and a CD8 transmembrane domain comprises a sequence having at least 80%, 85%, 90%, or 95% sequence identity with SEQ ID NO: 16 In some constructs the amino acid sequence of the porcine 4-1BB signaling domain comprises an amino acid sequence having at least 80%, 85%, 90%, or 95% sequence identity with SEQ ID NO: 24. In some constructs the amino acid sequence of the porcine CD3 zeta signaling domain comprises an amino acid sequence having at least 80%, 85%, 90%, or 95% sequence identity with SEQ ID NO: 25.

Methods described herein can further comprise introducing a nucleic acid encoding a heterologous polypeptide into the ungulate T cell or population of ungulate T cells. The heterologous polypeptide is optionally selected from the group consisting of CD46, CD55 and CD59. Methods described herein can further comprise incorporating the population of ungulate cells (e.g., porcine cells) into a pharmaceutical composition.

The present invention also serves to overcome current challenges in oncolytic virotherapy by providing a source of cells less likely to be rejected by a recipient. By way of example, an ungulate spleen provides such a source when the ungulate is genetically modified to eliminate or reduce antigens recognized by a recipient. Cells derived from the genetically modified ungulate spleen can be infected with the oncolytic virus, with or without further genetic modification, and used as a delivery system for the oncolytic virus. The cells of the transgenic animals can be engineered to evade hyperacute rejection. Further genetic modifications can be designed to limit T-cell mediated responses and natural killer (NK) cell responses and/or to target the cells to tumor cells.

Thus, provided herein is a genetically modified ungulate cell (e.g., from transgenic porcine, ovine or bovine animal) comprising an oncolytic virus, wherein the cell is derived from a transgenic ungulate. The transgenic ungulate cells provided herein can be derived from a transgenic ungulate genetically modified to reduce rejection of a cell or tissue derived from the transgenic animal when transplanted into a human host. The ungulate cell can be selected from the group consisting of a porcine, ovine or bovine cell.

The transgenic ungulates can be genetically modified to reduce expression of alpha 1,3 galactosyltransferase; beta-2-microglobulin; T cell receptor alpha; T cell receptor beta; or class II, major histocompatibility complex, transactivator (CIITA). Optionally, the expression of alpha 1,3 galactosyltransferase; beta-2-microglobulin; T cell receptor alpha, T cell receptor beta, or class II, major histocompatibility complex, transactivator (CIITA) is conditionally reduced. In some cases, the transgenic ungulate is genetically modified to heterologously express a HLA-E-beta2M single chain trimer.

In some genetically modified cells, the oncolytic virus is selected from the group consisting of vesicular stomatitis virus, measles, adenovirus, reovirus, herpes simplex virus, coxsackievirus A21, and vaccinia.

As described herein, the genetically modified, ungulate cell can selected from the group consisting of a monocyte, T lymphocyte, B lymphocyte, and natural killer (NK) cell. Optionally, the cell is further genetically modified to enhance delivery of the oncolytic virus to a tumor cell. Optionally, the genetically modified, ungulate cell is genetically modified to express a receptor for the oncolytic virus.

Optionally, any of the genetically modified ungulate cells provided herein can be genetically modified to disrupt expression of one or more proteins that prevent viral infection in an ungulate. Toll-like receptor (TLR), retinoic acid inducible gene-1 (RIG-1) and IFN-mediated antiviral response are three antiviral innate pathways. The genomes of some viruses are detected by members of the TLR family of receptors, which have a single transmembrane domain and recognize their ligands through leucine rich repeats in their luminal domains. The cytoplasmic toll/IL-1 receptor (TIR) domain of these receptors enable recruitment of adaptors such as TRIF or MyD88 that signal to downstream transcription factors. For the RIG-1 pathway, the C-terminal helicase domain of RIG-1 binds dsRNA and activated N-terminal CARD domains that initiate the downstream signaling cascade. For IFN, each IFN receptor (IFNAR1 and IFNAR2) subunit binds constitutively to a single specific member of the Janus kinase (JAK) family: IFNAR1 to tyrosine kinase 2 (TYK2) and IFNAR2 to JAK1. Type I IFN binding induces the phosphorylation of JAK1, TYK2, intracellular tyrosine residues of each receptor chain and signal transducers and activators of transcription (STATs). Activated STATs dimerize, dissociate from the receptor and together with IRF-9 (ISGF3 complex) translocate to the nucleus to induce the expression of more than 300 IFN-stimulated genes (ISGs). Disrupting expression of any one or more of the proteins along these pathways enhances prevention of viral infection.

Any of the genetically modified ungulate cells described herein can be genetically modified to express a chimeric antigen receptor (CAR). For example, the CAR can comprise a human antigen binding domain operably linked to one or more porcine intracellular signaling domains. In some genetically modified ungulate cells, the CAR is an anti-CD19 CAR, an anti-BCMA CAR, an anti-HER-2 CAR, an anti-EGFR CAR, or an anti-prostrate specific membrane antigen (PSMA) CAR. In some genetically modified ungulate cells, the CAR is a single chain variable fragment (scFv). In some genetically modified ungulate cells, the cell heterologously expresses CD46 complement regulatory protein (CD46), complement decay-accelerating factor (CD55), or MAC-inhibitory protein (CD59).

Also provided is a genetically modified ungulate cell comprising a chimeric antigen receptor (CAR), wherein the cell is derived from a transgenic ungulate. The cell can be derived from a transgenic ungulate genetically modified to reduce rejection of a cell or tissue derived from the transgenic animal when transplanted into a human host. In some cases, the ungulate cell is selected from the group consisting of a porcine, ovine or bovine cell. In some examples, the cell is derived from a transgenic ungulate, wherein the transgenic ungulate is genetically modified to express a CAR and genetically modified to reduce rejection of a cell or tissue derived from the transgenic animal when transplanted into a human host. To reduce rejection of a cell or tissue derived from the transgenic animal, the transgenic ungulate is genetically modified to reduce expression of alpha 1,3 galactosyltransferase; beta-2-microglobulin; one or more polypeptides of the T cell receptor complex; or class II, major histocompatibility complex, transactivator (CIITA). In some examples, the expression of alpha 1,3 galactosyltransferase; beta-2-microglobulin; one or more polypeptides of the T cell receptor complex; or class II, major histocompatibility complex, transactivator (CIITA) is conditionally reduced. In some cases, the transgenic ungulate is genetically modified to heterologously express a HLA-E-beta2M single chain trimer.

Any of the genetically modified CAR ungulate cells provided herein can further comprise an oncolytic virus. In any of the genetically modified, CAR ungulate cells provided herein, the oncolytic virus can be selected from the group consisting of vesicular stomatitis virus, measles, coxsackievirus A21, adenovirus, reovirus, herpes simplex virus, and vaccinia.

In some examples, the genetically modified CAR ungulate cell is a T lymphocyte or a natural killer (NK) cell. In some examples, the cell is a T lymphocyte. Also provided is a population comprising one or more genetically modified ungulate cells described herein.

Also provided is a pharmaceutical composition comprising any transgenic, ungulate cell described herein or a population of transgenic ungulate cells described herein and a pharmaceutically acceptable carrier.

Further provided is a transgenic ungulate comprising two or more genetic modifications to reduce rejection of a cell or tissue derived from the transgenic ungulate when transplanted into a human host. The modifications are optionally selected from the group consisting of a modification that reduces expression of alpha 1,3 galactosyltransferase; a modification that reduces expression of beta-2-microglobulin; a modification that reduces expression of T cell receptor alpha; a modification that reduces expression of T cell receptor beta; a modification that reduces expression of class II, major histocompatibility complex; a modification that reduces expression of transactivator (CIITA); and an insertion of a nucleic acid sequence encoding a HLA-E-beta2M single chain trimer. Optionally, the ungulate is further modified such that cells of the ungulate comprise a nucleic acid sequence encoding a heterologous polypeptide.

By way of example, any of the transgenic ungulates provided herein can further comprise a heterologous nucleic acid sequence encoding a CAR. By way of example, the heterologous polypeptide can be selected from the group consisting of CD46, CD55 and CD59.

Also provided is a method for treating cancer comprising administering to a subject having cancer a transgenic ungulate cell described herein, a population of genetically modified ungulate cells described herein, or a pharmaceutical composition described herein. In some methods, the cancer comprises a solid tumor. In some methods, the cancer is a hematological cancer. In some methods, the hematological cancer comprises a CD19-expressing cancer. Some methods further comprise administering a second anticancer therapy to the subject, such as a chemotherapy, immunotherapy, hormone therapy, cytokine therapy, radiotherapy, cryotherapy, or surgical therapy.

Also provided is a method for producing a genetically modified, ungulate cell as a carrier for an oncolytic virus, comprising obtaining a cell or population of cells from a transgenic ungulate that is genetically modified to reduce rejection of a cell or tissue derived from the transgenic ungulate when transplanted into a human host and infecting the cell or population of cells with an oncolytic virus.

Also provided is a method for producing a genetically modified, ungulate cell expressing a chimeric antigen receptor (CAR), comprising introducing in vitro a nucleic acid encoding the CAR into a cell or population of cells derived from a transgenic ungulate genetically modified to reduce rejection of a cell or tissue derived from the transgenic animal when transplanted into a human host or comprising obtaining a cell or population of cells from a transgenic ungulate that is genetically modified to express a CAR and genetically modified to reduce rejection of a cell or tissue derived from the transgenic ungulate when transplanted into a human host.

Some methods further comprise introducing a nucleic acid encoding a heterologous polypeptide into the cell or population of cells derived from the transgenic ungulate. In such methods, the heterologous polypeptide is selected from the group consisting of CD46, CD55 and CD59.

Optionally, the methods comprise isolating one or more T lymphocytes, B lymphocytes, natural killer (NK) cells, or monocytes. Some methods further comprise expanding the population of the selected genetically modified, ungulate cells. Some methods further comprising infecting the cell or population of cells with an oncolytic virus (e.g., vesicular stomatitis virus, measles, adenovirus, reovirus, herpes simplex virus, coxsackievirus A21, or vaccinia). Optionally the population of cells is incorporated into a pharmaceutical composition.

### DESCRIPTION OF THE FIGURES

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIG. 1A shows that porcine PBMCs contains 45-55% of CD3⁺ T cells according to flow analysis (FIG. 1A).

FIG. 1B shows that the removal of monocytes and B cells by negative selection increased the purify of T cells in the population of porcine PBMCs to 70-85%.

FIG. 2A shows that PHA is the strongest stimulator of porcine T cells, followed by PMA in combination with anti-porcine CD3 antibodies. Anti-porcine CD3 antibodies alone or in combination with human or mouse CD28 antibodies did not activate T cells.

FIG. 2B is an exemplary schematic of a lentiviral vector construct comprising a CD19scFV This construct comprises a nucleic acid sequence encoding CD19scFv, a CD8 hinge region, a CD8-transmembrane region, porcine 4-11B, porcine CD3 zeta (SEQ ID NO: 2). This exemplary construct is operably linked to an EF1α promoter sequence and a CMV enhancer sequence. The construct further comprises a nucleic acid sequence encoding a self-cleaving peptide (P2A) and a selectable marker (e.g., EmGFP).

FIG. 2C shows that only 1% of PHA-stimulated T cells was GFP⁺, while PMA plus CD3 antibody stimulated T cells were not transduced by lentivirus.

FIG. 3A shows exemplary constructs that can be transduced into K562 cells to make artificial dendritic cells. SEQ ID NO: 3 comprises a nucleic acid sequence encoding porcine CD80, a self-cleaving sequence (P2A), myc and porcine CD83. SEQ ID NO: 4 comprises a nucleic acid sequence encoding human CD64, a self-cleaving peptide sequence (P2A) and porcine 41BBL. SEQ ID NO: 5 comprises a nucleic acid sequence encoding an anti-porcine CD3 scFv and an mCD80 transmembrane region.

FIG. 3B shows the results of co-incubation experiments. K562 cells stably expressing CD80 and CD83 were transduced either with lentiviral-encoded membrane-bound scCD3 antibodies (K-DC2) or with LV encoded CD64 and 4-1BBL (K-DC1). Analysis of T cell proliferation after 4 days of co-incubation with K-DC1 or K-DC2, showed that K-DC2 are superior in induction of T cells proliferation, while K-DC1 were less efficient and induced only moderate proliferation.

FIG. 4A provides further analysis of T cell activation and proliferation over nine days, after initial stimulation with K-DC2. Activation was estimated by an increase in CD25 expression on the surface of T cells. Maximum activation was observed at day three

FIG. 4B shows flow analysis of CD25 expression on the surface of CD8+ and CD4+ T cells three days after activation with K-DC2. CD25 expression increased more than 10 times compared to non-stimulated cells

FIG. 4C shows that stimulation with K-DC2 dramatically increased proliferation of T cells compared to non-stimulated T cells

FIG. 5A shows CAR-T cells expansion after lentiviral transduction. The transduction level of porcine T cells on retronectin-coated plates is better than on standard uncoated plates. Data is shown for CAR-T cells, 11 days post transduction.

FIG. 5b shows an increase in the levels of CAR-T cells in T cell culture during the expansion period.

FIG. 5C shows flow analysis and microscope images of CAR-T cells (the same cells of FIG. 5A), expanded for 14 days after transduction. This figure shows that more than 50% of T cells in culture are CAR positive.

FIG. 6A shows the functional activity and specificity of CAR-T cells. Porcine hCD19 CAR T (effector cells) specifically killed the target CD19⁺NALM6/F-Luc cells in comparison to T cells transduced with mock (GFP) virus. At the same time, both hCD19 CAR T and mock-transduced porcine T cells had very little killing activity against CD19-K562 cells FIGS. 6B and C show that porcine hCD19 CAR T cells incubated with target CD19⁺NALM6 cells produce high levels of IFNγ compared to incubation with CD19⁻K562 cells. Mock (GFP) transduced T cells were not activated by CD19⁺NALM6 cells

FIG. 7 is an exemplary schematic illustrating the advantages of using the transgenic ungulate cells provided herein as carriers for oncolytic viruses for treating cancer in a subject.

FIG. 8 is an exemplary schematic for circumventing antiviral antibodies using transgenic ungulate carrier cells as delivery vehicles for an oncolytic measles virus.

FIG. 9 is an exemplary schematic for circumventing VSV antibodies and treating a human cancer using VSV-infected CAR-expressing pig T cells engineered to evade hyperacute rejection in a human.

FIG. 10 is an exemplary schematic for the treatment of a human cancer with CAR-expressing pig T cells engineered to evade hyperacute rejection in a human.

FIG. 11 is an exemplary schematic for the treatment of a human cancer using VSV-infected, CAR-expressing pig T cells engineered to evade hyperacute rejection in a human.

FIG. 12 is a schematic of an exemplary construct of a CAR comprising a CD19scFv, a CD8 hinge region, and a transmembrane-cytoplasmic signaling domain comprising motifs from pig CD3zeta, and pig 4-1BB. This construct comprises a nucleic acid sequence encoding a CD8 α leader sequence, a CD19scFv, a CD8α hinge region, a CD8α-transmembrane region, porcine 4-1BB, and porcine CD3 zeta.

### DETAILED DESCRIPTION

Provided herein are genetically modified ungulate cells comprising a nucleic acid encoding a chimeric antigen receptor (CAR). Also provided are methods of making these cells and methods of treating cancer by administering these cells to a subject in need thereof.

Also provided herein are transgenic ungulate cells that can be used as cell carriers for the delivery of oncolytic viruses in cancer therapy. Also provided are transgenic ungulates comprising these cells, methods of making these cells, and methods of treating cancer by administering these cells to a subject in need thereof. By using a transgenic ungulate cell that has been genetically modified to render the cell less immunogenic and/or less likely to induce a hyperacute or acute rejection reaction as a carrier, the oncolytic virus can be reliably delivered to tumor cells at sites of tumor growth. In addition, these carrier cells can protect the virus from antibodies and complement; extravasate from tumor neovessels and enter into the tumor parenchyma (trafficking); and/or transfer progeny viruses to tumor cells. In some cases, the transgenic ungulate cells described herein express a chimeric antigen receptor, thus providing intrinsic antitumor activity to the cell.

### Genetically Modified Ungulate Cells

Provided herein is a genetically modified ungulate cell(s) comprising a nucleic acid that encodes a chimeric antigen receptor (CAR). As used throughout, an ungulate is a hoofed mammal, for example, a porcine, bovine or ovine animal. Thus, any of the cells described herein can be selected from the group consisting of a porcine (pig), ovine (sheep) or bovine (cow) cell.

As used throughout, the term genetically modified ungulate cell refers to a cell derived from a genetically modified (i.e., transgenic) ungulate, or a cell that is derived from a non-genetically modified (i.e., non-transgenic) ungulate and subsequently genetically modified (e.g., genetically modified to comprise a nucleic acid encoding a CAR). As used herein, the term derived from an ungulate means obtained from a transgenic or non-transgenic ungulate. It is understood that genetically modified cells derived from transgenic ungulates can be further modified in vitro to comprise one or more additional genetic modifications (e.g., a nucleic acid encoding a CAR) as described herein.

Also provided herein is a genetically modified ungulate cell comprising an oncolytic virus, wherein the cell is derived from a transgenic ungulate. Any of the genetically modified cells derived from a transgenic ungulate can be selected from the group consisting of a porcine (pig), ovine (sheep) or bovine (cow) cell. The cell can be obtained from the blood, an organ or a tissue of the transgenic ungulate, without limitation.

As described throughout, the genetically modified ungulate cell can be from a transgenic ungulate genetically modified to reduce rejection of a cell or tissue derived from the transgenic animal when transplanted into a human host. In some examples, the transgenic ungulate is genetically modified to reduce expression of an antigen that causes hyperacute rejection upon transplantation of a cell derived from the transgenic ungulate into a human host. In some examples, the transgenic ungulate is genetically modified to reduce expression of alpha 1,3 galactosyltransferase (encoded by the GGTA1 gene), cytidine monophospho-N-acetylneuraminic acid hydroxylase (encoded by the CMAH gene) and/or polypeptide N-acetylgalactosaminyltransferase 2 (encoded by the GALNT2 gene) (i.e., a triple knockout or triple KO ungulate). Methods for producing transgenic ungulates that are genetically modified to reduce expression of alpha 1,3 galactosyltransferase, cytidine monophospho-N-acetylneuraminic acid hydroxylase and/or polypeptide N-acetylgalactosaminyltransferase 2 are known in the art. See, for example, U.S. Pat. Nos. 9,006,510, 10,912,863, and 7,795,493, 10,667,500 and U.S. Patent Application Publication No. 20170311579.

In some cases, expression of beta-2-microglobulin is reduced to decrease MHC class 1 expression. Reducing the expression of beta-2-microglobulin prevents recognition of the transgenic ungulate cells by recipient T cells that may interact with the MCH class 1-peptide complexes on the transgenic ungulate cells and eliminate them. In other cases, expression of class II, major histocompatibility complex, transactivator (CIITA) is reduced to decrease MHC class 2 expression. This prevents recognition of the transgenic ungulate cells by recipient T cells that may interact with the MHC class 2-peptide complexes on the transgenic ungulate cells and eliminate them.

The transgenic ungulate cells can also be engineered to prevent graft-versus-host disease in a recipient. This can be achieved by reducing expression of one or more polypeptides of the T cell receptor (TCR) complex, for example, by reducing expression of T cell receptor alpha and/or T cell receptor beta. The TRAC gene locus, which regulates expression of the T cell receptor alpha and/or the TRBC locus which regulates expression of T cell receptor beta chain can be modified to reduce expression of TCR-alpha and TCR-beta, respectively.

In some cases, the transgenic ungulate is modified by knocking in or heterologously expressing one or more polypeptides, for example, a HLA-E-beta2M single chain trimer. This modification prevents NK cell recognition and elimination of MHC negative cells in the recipient upon transplantation of the transgenic ungulate cells.

As used throughout, heterologous refers to what is not normally found in nature. For example, a heterologous nucleotide sequence encoding a heterologous polypeptide refers to a nucleotide sequence not normally found in a given cell in nature. As such, a heterologous nucleotide sequence may be foreign to its host cell (i.e., is exogenous to the cell); naturally found in the host cell (i.e., endogenous) but present at an unnatural quantity in the cell (i.e., greater or lesser quantity than naturally found in the host cell); or be naturally found in the host cell but positioned outside of its natural locus.

As used throughout, the terms polypeptide, peptide, and protein are used interchangeably herein to refer to a polymer of amino acid residues. The terms encompass amino acid chains of any length, including full-length proteins, wherein the amino acid residues are linked by covalent peptide bonds.

As used throughout, the term nucleic acid or nucleotide refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Any of the nucleic acid sequences described herein can also be codon-optimized.

As used throughout, the term knockout or KO, refers to an ungulate with reduced expression of a protein, for example, alpha-1,3-galactosyltransferase expression, cytidine monophospho-N-acetylneuraminic acid hydroxylase expression, polypeptide N-acetylgalactosaminyltransferase 2 expression, beta-2-microglobulin expression, TCR-alpha expression, TCR-beta expression or CIITA (for example, reduced mRNA and/or protein expression), such that the ungulate does not produce functional alpha-1,3-galactosyltransferase, beta-2-microglobulin, TCR-alpha, TCR-beta or CIITA. Optionally, the gene encoding alpha-1,3-galactosyltransferase, cytidine monophospho-N-acetylneuraminic acid hydroxylase, polypeptide N-acetylgalactosaminyltransferase 2, beta-2-microglobulin, TCR-alpha, TCR-beta or CIITA is modified such that no transcription of the gene occurs. Optionally, the gene encoding alpha-1,3-galactosyltransferase, cytidine monophospho-N-acetylneuraminic acid hydroxylase, polypeptide N-acetylgalactosaminyltransferase 2, beta-2-microglobulin, TCR-alpha, TCR-beta or CIITA is modified such that no translation of the gene occurs.

As used herein, the term gene refers to a segment of DNA involved in producing or encoding a polypeptide chain. It may include regions preceding and following the coding region (leader and trailer) as well as intervening sequences (introns) between individual coding segments (exons). It is understood that a reduction in gene expression need not be complete, as the expression level of a gene can be reduced or decreased to varying degrees. For example, expression can be reduced by 100%, 99%, 95%, 90%, 85%, 80%, or 75% as compared to expression in an unmodified control.

A gene encoding alpha-1,3-galactosyltransferase, cytidine monophospho-N-acetylneuraminic acid hydroxylase, polypeptide N-acetylgalactosaminyltransferase 2, beta-2-microglobulin, TCR-alpha, TCR-beta or CIITA can be genetically modified using any method known in the art. For example, the gene can be modified by deletion, substitution, mutation, insertion, rearrangement, or a combination thereof. The modification can disrupt the gene such that transcription and/or translation of the gene is reduced. By way of example, an insertion can generate a stop codon in the middle of a gene, or shift the open reading from of the gene. In the transgenic ungulates described herein, one or both alleles of a gene encoding alpha-1,3-galactosyltransferase, cytidine monophospho-N-acetylneuraminic acid hydroxylase, polypeptide N-acetylgalactosaminyltransferase 2, beta-2-microglobulin, TCR-alpha, TCR-beta or CIITA can be knocked out or inactivated. Therefore, homozygous knockouts where both alleles are inactivated as well as heterozygous knockouts where one allele is inactivated are provided herein.

Methods for making knockout transgenic animals, include, but are not limited to, oocyte pronuclear DNA microinjection, intracytoplasmic sperm injection, embryonic stem cell manipulation, somatic nuclear transfer, recombinase systems (for example, Cre-LoxP systems, Flp-FRT systems and others), zygote micromanipulation, zinc finger nucleases (ZNFs), transcriptional activator-like effector nucleases (TALENs), meganucleases, and RNA-guided gene editing systems (for example, clustered regularly interspaced short palindromic repeat/CRISPR/Cas systems, such as CRISPR/Cas9)). See, for example, Perisse et al. “Improvements in Gene Editing Technology Boost Its Applications in Livestock. Front Genet.” 2020; 11:614688. Epub 2021/02/20. doi: 10.3389/fgene.2020.614688; and Polejaeva, 25th Anniversary of Cloning by Somatic Cell Nuclear Transfer: Generation of genetically engineered livestock using somatic cell nuclear transfer. Reprod Suppl. 2021; 162 (1):F11-F22. Epub 2021/05/28. doi: 10.1530/REP-21-0072).

In some methods, a genetic modification is made to a somatic cell, for example, via CRISPR/Cas, TALENS, meganuclease technology, etc., and the nucleus of the somatic cell is transferred to an enucleated egg of the same species. In some methods, the enucleated eggs or oocytes are used for somatic cell nuclear transfer and are then transferred to a surrogate mother. In some embodiments, genetically modified zygotes are transferred to a surrogate mother.

As used herein, the term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. A genetic modification made by TALENs or other tools may be, for example, chosen from the list consisting of an insertion, a deletion, insertion of an exogenous nucleic acid fragment, and a substitution. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence.

In some methods, a TALEN-pair is introduced into a livestock cell or embryo that makes a genetic modification to DNA of the cell or embryo at a site that is specifically bound by the TALEN-pair, to the livestock animal from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods.

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes. Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

The CRISPR/Cas system, an RNA-guided nuclease system that employs a Cas endonuclease, can be used to edit the genome of a host cell or organism. The CRISPR/Cas system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, the term Cas9 refers to an RNA-mediated nuclease (e.g., of bacterial or archaeal origin or derived from a bacterial or archaeal nuclease). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p 759-771, 22 Oct. 2015), Cas13-based RNA editors, Cas-CLOVER (Li et al., Cas-CLOVER™: A High-Fidelity Genome Editing System for Safe and Efficient Modification of Cells for Immunotherapy. 2018 Precision CRISPR Congress Poster Presentation, Boston, MA) and homologs thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chloroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi et al., RNA Biol. 2013 May 1; 10(5): 726-737; Makarova et al., Nat. Rev. Microbiol. 2011 June; 9(6): 467-477; Hou et al., Proc Natl Acad Sci USA 2013 Sep. 24; 110(39):15644-9; Sampson et al., Nature, 2013 May 9; 497(7448):254-7; and Jinek et al., Science 2012 Aug. 17; 337(6096):816-21. Variants of any of the Cas9 nucleases provided herein can be optimized for efficient activity or enhanced stability in the host cell. Thus, engineered Cas9 nucleases are also contemplated. See, for example, Slaymaker et al., Rationally engineered Cas9 nucleases with improved specificity, Science 351 (6268): 84-88 (2016)).

Founder animals (F0 generation) can be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that the cells in their genome have undergone modification. Founders can be mosaic for a modification, as may happen when vectors are introduced into one of a plurality of cells in an embryo, typically after initial embryo cleavage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

The knockout animals described herein include both progenitor and progeny animals. Progeny animals include animals that are descended from the progenitor as a result of sexual reproduction or cloning and that have inherited genetic material from the progenitor. Thus, the progeny animals comprise the genetic modification introduced into the parent or other predecessor. A knockout animal may be developed, for example, from embryonic cells into which the genetic modification has been directly introduced or from the progeny of such cells. Animals that are produced by transfer of genetic modification (i.e., a gene knockout) through breeding of the animal comprising the genetic modification are also included. A cell or a population of cells from any of the knockout animals provided herein is also provided.

A gene knockout can be in any cell, organ, and/or tissue in an ungulate. The knockout can be a whole body knockout, e.g., expression of a gene is reduced in all cells of an ungulate. Knockout can also be specific to one or more cells, tissues, and/or organs of an ungulate. This can be achieved by conditional knockout, where expression of a gene is selectively reduced in one or more organs, tissues or types of cells. In some transgenic ungulate animals described herein, the expression of alpha-1,3-galactosyltransferase expression, cytidine monophospho-N-acetylneuraminic acid hydroxylase expression, polypeptide N-acetylgalactosaminyltransferase 2 expression, beta-2-microglobulin expression, T cell receptor alpha, T cell receptor beta, or class II, major histocompatibility complex, transactivator (CIITA) is conditionally reduced. Conditional knockout can be performed by a Cre-lox system, where cre is expressed under the control of a cell, tissue, and/or organ specific promoter. For example, a gene can be knocked out (or expression can be reduced) in one or more tissues, or organs, where the one or more tissues or organs can include brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary, testis, and/or any combination thereof.

Conditional knockouts can be inducible, for example, by using tetracycline inducible promoters or development specific promoters. This allows for elimination or suppression of gene/protein expression at any time or at a specific time. For example, with the case of a tetracycline inducible promoter, tetracycline can be given to an ungulate any time after birth. In some instances, the promoter can be induced by giving tetracycline to the mother during pregnancy. Once tetracycline is given to the ungulate, the tetracycline will result in expression of cre, which will then result in excision of a gene of interest.

A cre/lox system can also be under the control of a developmental specific promoter. For example, some promoters are turned on after birth or even after the onset of puberty. These promoters can be used to control cre expression and therefore can be used in developmental specific knockouts. See, for example, Chen et al., Construction of transgenic swine with induced expression of Cre recombinase, Animal 4(5), 767-771 (2010).

The tetracycline-inducible system and the Cre/loxP recombinase system (either constitutive or inducible) are commonly used inducible systems. The tetracycline-inducible system involves a tetracycline-controlled transactivator (tTA)/reverse tTA (rtTA). In vivo use of these systems involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two sets of animals provides control of gene expression.

Some transgenic ungulates can comprise a conditional knockout of the beta2 microglobulin gene by insertion of two spatially separated non-inhibitory loxP sites into the gene and insertion of a drug activatable (e.g., tetracycline responsive) expression cassette for Cre recombinase such that the gene fragment between the two loxP sites can be removed at a desired time (for example, after the cells of interest have been harvested from the animal and before they are used for therapy) by adding the corresponding drug (e.g., tetracycline) to the cells to activate expression of the cre recombinase gene. Expression of Cre recombinase leads to loxP site recombination and intervening gene fragment excision. This conditional knockout is useful for expression of MHC class 1 molecules, to ensure correct development of the T cell lineage in the genetically modified animal.

Combinations of knockout technology can also be used. For example, tissue specific knockout can be combined with inducible technology, creating a tissue specific, inducible knockout. Furthermore, other systems such as a developmental specific promoter can be used in combination with tissue specific promoters and/or inducible knockouts.

Any of the genetically modified ungulate cells provided herein can be selected from the group consisting of a monocyte, T lymphocyte, B lymphocyte, macrophage, mesenchymal stem cell, dendritic cell, bone marrow-derive stem/stromal cell (BMSC), and natural killer (NK) cell. In some cases, the cells are peripheral blood mononuclear cells, for example, unsorted PBMCs obtained from the transgenic ungulate.

Some genetically modified ungulate cells are T lymphocytes (T cells) or cells capable of differentiating into T cells, for example, cells that expresses a TCR receptor molecule. These include hematopoietic stem cells and cells derived from hematopoietic stem cells. As used herein, hematopoietic stem cell refers to a type of stem cell that can give rise to a blood cell. Hematopoietic stem cells can give rise to cells of the myeloid or lymphoid lineages or a combination thereof. Hematopoietic stem cells are predominantly found in the bone marrow, although they can be isolated from peripheral blood or a fraction thereof or from the spleen. As used herein, hematopoietic cell refers to a cell derived from a hematopoietic stem cell. The hematopoietic cell may be obtained or provided by isolation from an organism, system, organ (e.g., spleen), or tissue (e.g., blood or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. The hematopoietic cell can also be an immune cell, such as a T cell, B cell, macrophage, a natural killer (NK) cell or dendritic cell.

Any of the cells described herein can be a primary cell. As used herein, a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used throughout, an oncolytic virus is a virus that selectively lyses cancer cells but does not lyse normal cells. In some cells, the oncolytic virus is selected from the group consisting of vesicular stomatitis virus (VSV), measles, adenovirus, reovirus, herpes simplex virus, gamma-herpes virus, Japan encephalitis (HVJ-E), reovirus, Seneca Valley virus, Maraba virus, New Castle Disease virus, foamy virus, varicella zoster virus, poxvirus, retrovirus, parvovirus, coxsackievirus A21, and vaccinia.

Some genetically modified ungulate cells are further genetically modified to enhance delivery of the oncolytic virus to a tumor cell. For example, the cell can be genetically modified to express a receptor for the oncolytic virus. Exemplary receptors for adenovirus include, but are not limited to, CD46, coxsackievirus and adenovirus receptor, sialic acid (SA), CD80/86, and heparan sulfate. Exemplary receptors for herpes simplex virus-1 include, but are not limited to, herpes virus entry mediator (HVEM), nectin, 3-O-sulfated heparan sulfate proteoglycan (3-OS-HS), paired immunoglobulin-like receptor α (PILRα), myelin-associated glycoprotein (MAG), non-muscle myosin heavy chain (NMHC)-IIA, αVβ3, αVβ5, αVβ6, and αVβ8. Exemplary receptors for gamma herpes virus include, but are not limited to, CD21, α5β1, MHC-II, and heparan sulfate. Exemplary receptors for Japanese encephalitis virus include, but are not limited to plasmalemma vesicle associated protein (PLVAP), and gastrokine3 (GKN3). An exemplary receptor for reovirus is al protein. Exemplary receptors for vaccinia virus include heparan sulfate and laminin. An exemplary receptor for Seneca Valley virus is anthrax toxin receptor 1. Exemplary receptors for coxsackievirus include, but are not limited to, CD55, ICAM-1, integrin αV β3, integrin αV β6, coxsackievirus and adenovirus receptor. An exemplary Maraba virus receptor is low-density lipoprotein receptor (LDLR). Exemplary receptors for measles virus include, but are not limited to, CD46, DC-SIGN, SLAMF1, and nectin 4. Heparan sulfate can be used as a receptor for foamy virus and varicella zoster. An exemplary receptor for pox virus is glycosaminoglycan. Exemplary receptors for retrovirus include, but are not limited to, Tvb, XPR1, SLC19A1 (Pit1), SLC20A2 (Pit2), FLVCR1, FeLIX, transferrin receptor (TFRC), SLC1A5. Exemplary receptors for VSV include CD46 and low-density lipoprotein receptor (LDLR). Exemplary receptors for New Castle Disease virus include, but are not limited to, 2,3 and 2,6 sialic acids. An exemplary receptor for parvovirus is integrin α5 β1.

Some cells are also genetically modified to disrupt expression of one or more proteins that prevent viral infection in an ungulate. Some cells are genetically modified to heterologously express CD46, CD55 or CD59.

Further provided is a genetically modified ungulate cell comprising a chimeric antigen receptor (CAR), wherein the cell is derived from a transgenic ungulate. The cell can be from a transgenic ungulate that comprises a heterologous nucleotide sequence encoding a CAR, or a cell obtained from a transgenic ungulate described herein that does not comprise a heterologous nucleotide sequence encoding a CAR, wherein the cell obtained from the transgenic ungulate is subsequently modified in vitro to express a CAR.

Some genetically modified ungulate CAR cells are from a transgenic ungulate genetically modified to reduce rejection of a cell or tissue derived from the transgenic animal when transplanted into a human host. Any of the transgenic ungulate CAR cells can be selected from the group consisting of a porcine, ovine or bovine cell. In some examples, the cell is derived from a transgenic ungulate, wherein the transgenic ungulate is genetically modified to express a CAR and genetically modified to reduce rejection of a cell or tissue derived from the transgenic animal when transplanted into a human host.

Any of the genetically modified ungulate CAR cells can be obtained from a transgenic ungulate genetically modified to reduce expression of alpha 1,3 galactosyltransferase, cytidine monophospho-N-acetylneuraminic acid hydroxylase polypeptide N-acetylgalactosaminyltransferase 2, beta-2-microglobulin, class II, major histocompatibility complex, transactivator (CIITA), T cell receptor alpha and/or T cell receptor beta, as described above. In some cases, the transgenic ungulate is modified by knocking in or heterologously expressing one or more polypeptides, for example, a HLA-E-beta2M single chain trimer, as described above.

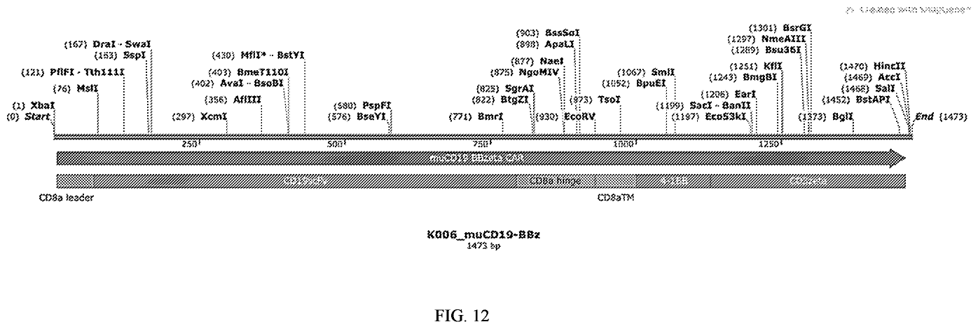

In any of the genetically modified CAR ungulate cells provided herein, the CAR can comprise a human antigen binding domain operably linked to one or more porcine intracellular signaling domains selected from the group consisting of CD3zeta, CD28 and 4-1BB. An exemplary construct, a nucleic acid encoding a CAR comprising a CD19scFv, a CD8 hinge region, and a transmembrane-cytoplasmic signaling domain comprising motifs from pig CD3zeta, and pig 4-1BB is set forth as SEQ ID NO: 1. A schematic of this construct is provided in FIG. 12. Another exemplary nucleic acid sequence comprising a CAR that includes a CD19scFv, a CD8 hinge region, and a transmembrane-cytoplasmic signaling domain comprising motifs from pig CD3 zeta, and pig 4-1BB is set forth as SEQ ID NO: 2. SEQ ID NO: 10 is an exemplary nucleic acid sequence encoding a human CD19scFv. SEQ ID NO: 11 is an exemplary nucleic acid sequence encoding a human optimized CD8α hinge transmembrane domain. SEQ ID NO: 12 is an exemplary sequence encoding a porcine 4-1BB signaling domain (NCBI Reference Sequence NM_001267073). SEQ ID NO: 13 is an exemplary sequence encoding a porcine CD3 zeta signaling domain (NCBI Reference Sequence AF15830.1).

Any of the constructs described herein can comprise a nucleic acid sequence having at least 70%, 80%, 90%, 95%, or 99% identity with a nucleic acid sequence comprising or consisting of SEQ ID NO: 1, 2, 3, 4, 5, 6, 7, 8, 9, 10, 11, 12 or 13. Any of the constructs described herein can comprise a nucleic acid sequence encoding an amino acid sequence having at least 70%, 80%, 90%, 95%, or 99% identity with an amino acid sequence comprising or consisting of SEQ ID NO: 14, 15, 16, 17, 18, 19, 20, 21, 22, 23, 24, or 25. Also provided are nucleic acid sequences comprising or consisting of a nucleic acid sequence having at least 70%, 80%, 90%, 95%, or 99% identity with a nucleic acid sequence comprising or consisting of SEQ ID NO: 1, 2, 3, 4, 5, 6, 7, 8, 9, 10, 11, 12 or 13. Also provided are polypeptides comprising or consisting of an amino acid sequence comprising or consisting of SEQ ID NO: 14, 15, 16, 17, 18, 19, 20, 21, 22, 23, 24, or 25.

The term identity or substantial identity, as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

Any of the constructs described herein can comprise one or more nucleic acid sequence encoding one or more self-cleaving peptides that separate the components of the construct. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al. “Cleavage efficient 2A peptides for high level monoclonal antibody expression in CHO cells,” MAbs 7(2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different.

In some embodiments, one or more linker sequences separate the components of any of the constructs described herein. The linker sequence can be two, three, four, five, six, seven, eight, nine, ten amino acids or greater in length.

As used herein, operably linked refers to placing one nucleic acid sequence or polypeptide into a functional relationship with another nucleic acid sequence or polypeptide. For example, a promoter or enhancer is operably linked to a coding sequence if it affects the transcription of the sequence; or a ribosome binding site is operably linked to a coding sequence if it is positioned so as to facilitate translation.

In any of the genetically modified CAR ungulate cells provided herein, the CAR be an anti-CD19 CAR, an anti-BCMA CAR, an anti-HER-2 CAR, an anti-EGFR CAR, an EGFRvIII CAR, an anti-mesothelin CAR, an anti-prostrate specific membrane antigen (PSMA) CAR, an anti-carcinoembryonic antigen (CEA) CAR, an anti-disialoganglioside 2 (GD2) CAR, an anti-IL-13Ra2 CAR, an anti-glypican-3 CAR, a anti-carbonic anhydrase IX (CAIX) CAR, an anti-L1 cell adhesion molecule (L1-CAM) CAR, an anti-cancer antigen 125 (CA125) CAR, an anti-fibroblast activation protein (FAP) CAR, an anti-cancer/testis antigen 1B (CTAG1B), an anti-mucin 1 CAR, an anti-folate receptor-a (FR-a) CAR, an anti-CD20 CAR or an anti-CD21 CAR. In any of the genetically modified CAR ungulate cells provided herein, the CAR can be a single chain variable fragment (scFv). See, for example, Dotti et al. “Design and Development of Therapies Using Chimeric Antigen Receptor-Expressing T cells,” Immunol. Rev. 257(1): 107-126 (2014).

Any of the genetically modified CAR ungulate cells provided herein can heterologously express C46, CD55 or CD59. CD46, CD55 and CD59 are human immune regulatory proteins. CD46 protects cells from complement-mediated attack, facilitates infection by a large number of pathogens and exerts complex effects on cellular immune function. CD55 or Decay-accelerating factor (DAF) inhibits the complement system. CD59 is a membrane-bound inhibitor of the terminal pathway of the complement cascade.

Any of the genetically modified CAR ungulate cells provided herein can further comprise an oncolytic virus. These include transgenic ungulate cells as well as non-transgenic ungulate cells that express a CAR. For example, the oncolytic virus can be selected from the group consisting of vesicular stomatitis virus (VSV), measles, adenovirus, reovirus, herpes simplex virus, gamma-herpes virus, Japan encephalitis (HVJ-E), reovirus, Seneca Valley virus, Maraba virus, New Castle Disease virus, foamy virus, varicella zoster virus, poxvirus, retrovirus, parvovirus, coxsackievirus A21, and vaccinia. It is understood that any genetically modified ungulate cell derived from a transgenic ungulate described herein, including CAR and non-CAR ungulate cells, can be infected with an oncolytic virus using methods known in the art.

The genetically modified cell obtained from an ungulate can be selected from the group consisting of a monocyte, T lymphocyte, B lymphocyte, macrophage, mesenchymal stem cell, dendritic cell, bone marrow-derive stem/stromal cell (BMSC), and natural killer (NK) cell. In some cases, the cells are peripheral blood mononuclear cells (PMBCs), for example, unsorted PBMCs, obtained from the transgenic ungulate.

Populations of any of the genetically modified CAR cells and non-CAR cells, including CAR cells and non-CAR cells, comprising an oncolytic virus as described herein, are also provided. Also provided is a pharmaceutical composition comprising any genetically modified ungulate cell described herein, including a transgenic ungulate cell described herein or a population of transgenic ungulate cells described herein and a pharmaceutically acceptable carrier.

### Transgenic Ungulates

Also provided are transgenic ungulates, for example, a transgenic porcine, ovine or bovine animal. For example, provided herein is a transgenic ungulate comprising two or more genetic modifications to reduce rejection of a cell or tissue derived from the transgenic ungulate when transplanted into a human host, wherein the modifications are selected from the group consisting of a modification that reduces expression of alpha 1,3 galactosyltransferase; a modification that reduces expression of beta-2-microglobulin; a modification that reduces expression of T cell receptor alpha; a modification that reduces expression of T cell receptor beta; a modification that reduces expression of class II, major histocompatibility complex; a modification that reduces expression of transactivator (CIITA); and an insertion of a nucleic acid sequence encoding a HLA-E-beta2M single chain trimer. Optionally, the transgenic ungulate further comprises a nucleic acid sequence encoding a heterologous polypeptide.

The transgenic ungulate can further comprise a genetic modification that reduces expression of cytidine monophospho-N-acetylneuraminic acid hydroxylase, and/or a genetic modification that reduces expression of polypeptide N-acetylgalactosaminyltransferase 2.

Optionally, the transgenic ungulate comprises a genetic modification that reduces expression of alpha 1,3 galactosyltransferase, a genetic modification that reduces expression of cytidine monophospho-N-acetylneuraminic acid hydroxylase and a genetic modification that reduces expression of polypeptide N-acetylgalactosaminyltransferase 2 (i.e., a triple knockout, or KO), and one or more genetic modifications selected from the group consisting of a modification that reduces expression of beta-2-microglobulin; a modification that reduces expression of T cell receptor alpha; a modification that reduces expression of T cell receptor beta; a modification that reduces expression of class II, major histocompatibility complex; a modification that reduces expression of transactivator (CIITA); and an insertion of a nucleic acid sequence encoding a HLA-E-beta2M single chain trimer.

In some transgenic ungulates, the expression of alpha-1,3-galactosyltransferase expression, cytidine monophospho-N-acetylneuraminic acid hydroxylase expression, polypeptide N-acetylgalactosaminyltransferase 2 expression, beta-2-microglobulin expression, TCR-alpha expression, TCR-beta expression or CIITA is conditionally reduced, as described above. Methods for making transgenic ungulates with genetic modifications are known in the art and described herein.

Some of the transgenic ungulates provided herein further comprise a nucleic acid sequence encoding a CAR. The transgenic ungulates optionally comprise a heterologous polypeptide selected from the group consisting of CD46, CD55 and CD59.

### Treatment Methods

Also provided is a method for treating cancer comprising administering to a subject having cancer any genetically modified or transgenic ungulate cell described herein; a population of genetically modified or transgenic ungulate cells described herein; or a pharmaceutical composition described herein.

Any of the genetically modified ungulate cells described herein (i.e., a non-CAR cell comprising an oncolytic virus, wherein the cell is derived from a transgenic ungulate; a CAR cell derived from a transgenic ungulate; a CAR cell produced by genetically modifying a cell from a non-transgenic ungulate; or a CAR cell comprising an oncolytic virus, wherein the CAR cell is derived from a transgenic ungulate), can be used to treat cancer in a subject in need thereof. As used herein, cancer is a disease characterized by the rapid and uncontrolled growth of aberrant cells. Cancer cells can spread locally or through the bloodstream and lymphatic system to other parts of the body. The cancer can be a solid tumor. In some cases, the cancer is a blood or hematological cancer, such as a leukemia (e.g., acute leukemia; acute lymphocytic leukemia; acute myelocytic leukemias, such as myeloblastic, promyelocytic, myelomonocytic, monocytic, erythroleukemia leukemias and myelodysplastic syndrome; chronic myelocytic (granulocytic) leukemia; chronic lymphocytic leukemia; hairy cell leukemia), polycythemia vera, or lymphoma (e.g., Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+anaplastic large-cell lymphoma (ALCL), acute myeloid lymphoma (AML), multiple myelomas (e.g., smoldering multiple myeloma, non-secretory myeloma, osteosclerotic myeloma, plasma cell leukemia, solitary plasmacytoma and extramedullary plasmacytoma), Waldenstrom's macroglobulinemia, monoclonal gammopathy of undetermined significance, benign monoclonal gammopathy and heavy chain disease.

Solid tumors include, by way of example, bone and connective tissue sarcomas (e.g., bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma), brain tumors (e.g., glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma), breast cancer (e.g., adenocarcinoma, lobular (small cell) carcinoma, intraductal carcinoma, medullary breast cancer, mucinous breast cancer, tubular breast cancer, papillary breast cancer, Paget's disease, and inflammatory breast cancer), adrenal cancer (e.g., pheochromocytoma and adrenocortical carcinoma), thyroid cancer (e.g., papillary or follicular thyroid cancer, medullary thyroid cancer and anaplastic thyroid cancer), pancreatic cancer (e.g., insulinoma, gastrinoma, glucagonoma, vipoma, somatostatin-secreting tumor, and carcinoid or islet cell tumor), pituitary cancers (e.g., Cushing's disease, prolactin-secreting tumor, acromegaly, and diabetes insipidus), eye cancers (e.g., ocular melanoma such as iris melanoma, choroidal melanoma, and ciliary body melanoma, and retinoblastoma), vaginal cancers (e.g., squamous cell carcinoma, adenocarcinoma, and melanoma), vulvar cancer (e.g., squamous cell carcinoma, melanoma, adenocarcinoma, basal cell carcinoma, sarcoma, and Paget's disease), cervical cancers (e.g., squamous cell carcinoma and adenocarcinoma), uterine cancers (e.g., endometrial carcinoma and uterine sarcoma), ovarian cancers (e.g., ovarian epithelial carcinoma, borderline tumor, germ cell tumor, and stromal tumor), esophageal cancers (e.g., squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma), stomach cancers (e.g., adenocarcinoma, fungating (polypoid), ulcerating, superficial spreading, diffusely spreading, malignant lymphoma, liposarcoma, fibrosarcoma, and carcinosarcoma), colon cancers, rectal cancers, liver cancers (e.g., hepatocellular carcinoma and hepatoblastoma), gallbladder cancers (e.g., adenocarcinoma), cholangiocarcinomas (papillary, nodular, and diffuse), lung cancers (e.g., non-small cell lung cancer, squamous cell carcinoma (epidermoid carcinoma), adenocarcinoma, large-cell carcinoma and small-cell lung cancer), testicular cancers (e.g., germinal tumor, seminoma, anaplastic, classic (typical), spermatocytic, nonseminoma, embryonal carcinoma, teratoma carcinoma, choriocarcinoma (yolk-sac tumor)), prostate cancers (e.g., adenocarcinoma, leiomyosarcoma, and rhabdomyosarcoma), penile cancers, oral cancers (e.g., squamous cell carcinoma), basal cancers, salivary gland cancers (e.g., adenocarcinoma, mucoepidermoid carcinoma, and adenoidcystic carcinoma), esopharyngeal cancers (e.g., squamous cell cancer and verrucous cancer), skin cancers (e.g., basal cell carcinoma, squamous cell carcinoma and melanoma, superficial spreading melanoma, nodular melanoma, lentigo malignant melanoma, acral lentiginous melanoma), kidney cancers (e.g., renal cell cancer, adenocarcinoma, hypemephroma, fibrosarcoma, transitional cell cancer (renal pelvis and/or ureter), Wilms' tumor), bladder cancers (e.g., transitional cell carcinoma, squamous cell cancer, adenocarcinoma, and carcinosarcoma). In addition, cancers include myxosarcoma, osteogenic sarcoma, endotheliosarcoma, lymphangio endothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

Some methods further comprise administering a second anticancer therapy to the subject. In some methods, the anticancer therapy is chemotherapy, immunotherapy, hormone therapy, cytokine therapy, radiotherapy, cryotherapy, or surgical therapy.

Table 1 provides targets (tumor antigens) for anti-tumor CAR therapy and the cancer that can be treated by targeting tumor antigen.

TABLE 1
Tumor antigens and Cancers

| | |
|---|---|
| CD19 | B-cell malignancies |
| EGFR | non-small cell lung cancer, |
| | epithelial carcinoma, and |
| | glioma |
| EGFRvIII | glioblastoma |
| HER2 | ovarian cancer, breast |
| | cancer, glioblastoma, colon |
| | cancer, osteosarcoma, and |
| | medulloblastoma |
| Mesothelin | mesothelioma, ovarian |
| | cancer, and pancreatic |
| | adenocarcinoma |
| Prostate-specific | prostate cancer |
| membrane antigen | |
| (PSMA) | |
| Carcinoembryonic | pancreatic adenocarcinoma, |
| antigen (CEA) | breast cancer and colorectal |
| | carcinoma |
| Disialoganglioside 2 | neuroblastoma and |
| (GD2) | melanoma |
| IL-13Ra2 | glioma |
| Glypican-3 | hepatocellular carcinoma |
| Carbonic anhydrase IX | renal cell carcinoma |
| (CAIX) | |
| L1 cell adhesion | neuroblastoma, melanoma |
| molecule (L1-CAM) | and ovarian adenocarcinoma |
| Cancer antigen 125 (CA | epithelial ovarian cancer |
| 125) | |
| CD133 | glioblastoma and |
| | cholangiocarcinoma |
| Fibroblast activation | malignant pleural |
| protein (FAP) | mesothelioma |
| Cancer/testis antigen 1B | melanoma and ovarian |
| (CTAG1B) | cancer |
| Mucin 1 | seminal vesicle cancer |
| Folate receptor-a (FR-a) | ovarian cancer |
| BCMA | myeloma |
| CD20 | B cell malignancies |
| CD21 | B cell malignancies |

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder. The term patient or subject includes human and veterinary subjects. In any of the methods provided herein, the subject can be a subject diagnosed with cancer.

As used herein the terms treatment, treat, or treating refers to a method of reducing one or more of the effects of the disorder or one or more symptoms of the disorder, for example, cancer in the subject. Thus in the disclosed methods, treatment can refer to a 10%, 20%, 30%, 40%, 50%, 60%, 70%, 80%, 90%, or 100% reduction in the severity of cancer. For example, a method for treating cancer is considered to be a treatment if there is a 10% reduction in one or more symptoms of the cancer in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

In the treatment methods described herein, the cells, population of cells, or pharmaceutical composition is administered in a therapeutically effective amount. As used herein, the term therapeutically effective amount or effective amount refers to an amount of a composition comprising any of the genetically modified ungulate cells described herein, or cells differentiated therefrom, that, when administered to a subject, is effective, alone or in combination with additional agents, to treat a disease or disorder either by one dose or over the course of multiple doses. A suitable dose can depend on a variety of factors including the particular cells used and whether they are used concomitantly with other therapeutic agents. Other factors affecting the dose administered to the subject include, e.g., the type or severity of the disease. For example, a subject having a leukemia may require administration of a different dosage of a composition comprising genetically modified ungulate cells described herein, or cells differentiated therefrom, than a subject with breast cancer.

The effective amount of genetically modified ungulate cells or cells differentiated therefrom can be determined by one of ordinary skill in the art and includes exemplary amounts for a mammal of about 0.1×10⁵ to about 8×10⁹ cells/kg of body weight. Other factors that influence dosage can include, e.g., other medical disorders concurrently or previously affecting the subject, the general health of the subject, the genetic disposition of the subject, diet, time of administration, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

As used herein, administer or administration refers to the act of introducing, injecting or otherwise physically delivering a substance as it exists outside the body (e.g., genetically modified ungulate cells, or cells differentiated therefrom) into a subject, such as by mucosal, intradermal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

Any of the genetically modified ungulate cells, cells differentiated therefrom, or second anticancer agents described herein are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539.

The cells described herein can be formulated as a pharmaceutical composition. Optionally, the pharmaceutical composition can further comprise a carrier. The term carrier means a compound, composition, substance, or structure that, when in combination with a compound or cells, aids or facilitates preparation, storage, administration, delivery, effectiveness, selectivity, or any other feature of the compound or cells for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

The genetically modified ungulate cells or cells differentiated therefrom can be formulated as a pharmaceutical composition for parenteral administration. In some examples, the pharmaceutical composition further comprises a second therapeutic agent. Optionally, the cells are typically administered in an aqueous solution, by parenteral injection.

### Methods of Making Genetically Modified Ungulate Cells

Also provided is a method for producing a genetically modified, ungulate cell as a carrier for an oncolytic virus, comprising obtaining a cell or population of cells from a transgenic ungulate that is genetically modified to reduce rejection of a cell or tissue derived from the transgenic ungulate when transplanted into a human host and infecting the cell or population of cells with an oncolytic virus. The cell can be, for example, a monocyte isolated from the spleen of a transgenic ungulate.

Also provided is a method for producing a genetically modified, ungulate cell expressing a chimeric antigen receptor (CAR), comprising introducing in vitro a nucleic acid encoding the CAR into a cell or population of cells derived from a transgenic ungulate genetically modified to reduce rejection of a cell or tissue derived from the transgenic animal when transplanted into a human host; or obtaining a cell or population of cells from a transgenic ungulate that is genetically modified to express a CAR and genetically modified to reduce rejection of a cell or tissue derived from the transgenic ungulate when transplanted into a human host.

Some methods further comprise introducing a nucleic acid encoding a heterologous polypeptide into the cell or population of cells derived from the transgenic ungulate. In some methods, the heterologous polypeptide is selected from the group consisting of CD46, CD55 and CD59.

In some methods, the cell is selected from the group consisting of a monocyte, T lymphocyte, B lymphocyte, macrophage, mesenchymal stem cell, dendritic cell, bone marrow-derive stem/stromal cell (BMSC), and natural killer (NK) cell. In some methods, the cells are peripheral blood mononuclear cells, for example, unsorted PBMCs obtained from the transgenic ungulate.

Some methods further comprise expanding the population of genetically modified, ungulate cells. Some methods further comprise differentiating the genetically modified, ungulate cells. Some methods further comprise infecting the cell or population of cells with an oncolytic virus. The oncolytic virus can be selected from the group consisting of vesicular stomatitis virus (VSV), measles, adenovirus, reovirus, herpes simplex virus, gamma-herpes virus, Japan encephalitis (HVJ-E), Seneca Valley virus, Maraba virus, New Castle Disease virus, foamy virus, varicella zoster virus, poxvirus, retrovirus, parvovirus, coxsackievirus A21, and vaccinia. Cells can be infected according to methods of Escobar-Zarate et a., Overcoming cancer cell resistance to VSV oncolysis with JAK1/2 inhibitors, Cancer Gene Ther. 2013 October; 20(10): 582-589. Some methods further comprise incorporating the population of cells into a pharmaceutical composition.

### Methods for Activating Ungulate T Cells and Uses Thereof

Further provided is a method for activating ungulate T cells comprising (a) obtaining a population of ungulate T cells and (b) contacting the population of ungulate T cells with a population of artificial antigen presenting cells, wherein the antigen presenting cells express ungulate CD80, ungulate CD83, and an anti-ungulate CD3 antibody. The T cells are optionally selected from the group consisting of porcine cells, ovine cells and bovine cells.

In any of the methods for activating ungulate T cells described herein, the population of ungulate T cells can be from any non-transgenic ungulate or from any transgenic ungulate described herein.

In some methods, the population of ungulate T cells is a population of porcine T cells. Optionally, the artificial antigen presenting cells (e.g., artificial dendritic cells as described in the Examples) express a porcine CD80 and a porcine CD83, and the anti-CD3 antibody is an anti-porcine CD3 antibody. The anti-CD3 antibody can be a scFv anti-CD3 antibody.

The population of ungulate cells (e.g., a population of porcine T cells) is optionally plated on a retronectin-coated dish in culture media comprising human IL-2 and ungulate IL-21 (e.g., porcine IL-21) prior to contacting the population of ungulate T cells with the artificial antigen presenting cells. The contacting step can comprise stimulating the population of ungulate T cells with the artificial antigen presenting cells at a ratio of about 5:1 (ungulate T cells: artificial antigen presenting cells), for example, a ratio of about 5:1 (porcine T cells: artificial antigen presenting cells)

Populations of the activated ungulate T cells described herein can be used to make a population of ungulate T cells that express a CAR.

Also provided is a method for making a population of genetically modified ungulate T cells comprising a chimeric antigen receptor (CAR) comprising (a) obtaining a population of activated ungulate T cells produced by any of the cell activation methods provided herein and (b) introducing a nucleic acid encoding the CAR into the population of ungulate T cells. In some methods, introducing the nucleic acid comprises transducing the population of activated ungulate T cells with a lentiviral vector comprising a nucleic acid construct encoding the CAR. The activated population of ungulate T cells can be a population of porcine T cells, which are optionally plated on retronectin-coated plates and which are optionally transduced in the presence of polybrene. The population of plated transduced porcine T cells is optionally expanded by culturing the population in culture media comprising human IL-2 and porcine IL-21. It is understood that these conditions can be used to transduce other ungulate cells with a construct encoding a CAR.

In some methods, the lentiviral vector comprises a nucleic acid encoding the CAR. The CAR optionally comprises a human antigen binding domain operably linked to one or more ungulate intracellular signaling domains. Optionally, the CAR is an anti-CD19 CAR, an anti-BCMA CAR, an anti-HER-2 CAR, an anti-EGFR CAR, or an anti-PSA CAR. The CAR can be a single chain variable fragment (scFv). Optionally, the lentiviral vector comprises a construct having (a) a nucleic acid sequence encoding an anti-CD19 scFv, (b) a nucleic acid encoding a CD8α hinge, (c) a CD8 transmembrane domain, (c) a porcine, cytoplasmic 41BB signaling domain, and (d) a cytoplasmic porcine CD3 zeta signaling domain, wherein the construct is operably linked to a promoter.

In some methods, the construct comprises a nucleic acid sequence having the nucleic acid sequence of SEQ ID NO: 1 or SEQ ID NO:2 or a nucleic acid sequence having at least 95% identity to SEQ ID NO: 1 or SEQ ID NO: 2. Optionally, the promoter is an EF1-α promoter.

Some methods further comprise introducing a nucleic acid encoding a heterologous polypeptide into the ungulate T cell or population of ungulate T cells. The heterologous polypeptide is optionally selected from the group consisting of CD46, CD55 and CD59. Some methods further comprise incorporating the population of ungulate cells (e.g., porcine cells) into a pharmaceutical composition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example I

### Ex Vivo Activation, Expansion, and Genetic Modification of Porcine T Cells

### Animals, Blood Collection and PBMC Isolation

Porcine whole blood was obtained from healthy pigs (Mayo Clinic, Rochester, MN) and kept on wet ice. To isolate peripheral blood mononuclear cells (PBMC), the Ficoll-Paque Plus gradient (Sigman Aldrich, St. Louis, MO) was used. Briefly, blood diluted with an equal volume of PBS was overlayed on Ficoll-Paque Plus and centrifuged at 1,300×g for 30 min at room temperature. The layer of PBMC formed on top of the Ficoll interface was collected and washed twice with PBS. These isolated PBMCs were used in subsequent steps for T cell or monocyte isolation. The cells can also be frozen in aliquots at 10 cells/vial in CryoStor CS10 media (Stemcell Technologies, Vancouver, CA) and stored in liquid nitrogen until the use.

### T Cells Isolation and Expansion

Porcine T cells were isolated by negative selection. Briefly, PBMC were first incubated with anti-porcine CD172a and CD21 antibodies and then with anti-mouse IgG microbeads (Miltenyi, Bergisch Gladbach, North Rhine-Westphalia, Germany). This antibody cocktail labels monocytes and B cells. To label and subsequently remove NK anti-porcine CD16 antibodies (Clone G7, BioRad, #MCA1971GA, Hercules, CA) can be added to the cocktail. After labeling, cells were applied to a magnetic column. The column was washed, and unlabeled T cells were collected as an unbound fraction.

Purified T cells were plated at a density of 2×10⁶ cells/ml in 10% FBS-AIM-V supplemented with 25 ng/ml hIL-2 and 10 ng/ml pIL-21 (TCM) and stimulated with mitomycin C-treated or γ-irradiated (120 Gy) artificial dendritic cells (K-DC2) at a 5:1 ratio (T cells: K-DC2). Cells were cultured for 3-4 days before transduction with LV.

### T Cell Transduction

Expanded and stimulated T cells were transduced with a lentiviral vector. i.e., LV-hCD19-CAR-GFP at a multiplicity of infection (MOI) of 1-10. Briefly, 3-4 days after stimulation T cells were collected from plate, washed once in PBS and cell pellet was re-suspended directly in AIM-V media mixed with LV and 8 μg/ml polybrene (PB). The final density of T cells during transduction varied between 1.25-2×10⁶/ml. After that, cells were immediately plated in 12- or 6-well plates. Sometimes, retronectin-coated plates were used. Plates were centrifuged at 800×g and 30° C. for 2 h and then placed in a CO₂ incubator. After two hours of incubation (total 4 hours after cells were mixed with LV), fresh 10% FBS-AIM-V was added to wells (half of well volume), and transduced T cells were continued to culture overnight. The next day, the media in the wells was completely changed to fresh 10% FBS-AIM-V supplemented with 50 ng/ml hIL-2 and 10 ng/ml pIL-21. Cells were cultured/expanded for an additional 4-14 days before analysis. The density of cells was maintained between 1.5-2.5×10⁶/ml.

### Retronectin-Coated Plates

The stock of Retronectin (1 mg/ml, Takara, Shiga, JP) was diluted with PBS to a concentration of 12.5 μg/ml. Plates were pre-coated with 12.5 μg/ml Retronectin overnight. The next day, plates were washed with PBS, and wells were blocked with 0.5% BSA in PBS for 1 h at room temperature. After washing with PBS, plates were ready to be used or were wrapped and stored at 4° C. for up to one week.

### Cell Media

Two types of media were used: RPMI (ATCC modification, at initial experiments) or AIM-V™ (ThermoFisher, Waltham, MA) (during later experiments). Both media were supplemented with 10% heat inactivated FBS and different cytokines.

### Additional Cytokines Used in Maintenance of T Cells

T cells should be stimulated and be able to proliferate well. Porcine IL-2 (50-10 U/ml) did not support T cells proliferation well. Therefore, different conditions were tested to find a reliable stimulation/proliferation protocol.

Combinations of cytokines which showed successful results in other animal models, for example, IL-2+IL-21 in dogs, were tested. (Panjwani, et al. “Establishing a model system for evaluating CAR T cell therapy using dogs with spontaneous diffuse large B cell lymphoma,” OncoImmunology, 9:1, DOI: 10.1080/2162402X.2019.1676615 (2020)). Recombinant IL-21 alone (Mata et al., Journal of Immunotherapy 37(8): 407-415 (1997)), rhIL-2 and rhIL-15 or, a combination of IL-15 and IL21 (Du et al. “Preparation Through Improving Lentivirus Mediated Transfection Efficiency of T Cells and Enhancing CAR-T Cell Cytotoxic Activities,” Frontiers in Molecular Biosciences, 8: Article 685179 (2021)) were used.

### Generation of Artificial Dendritic Cells

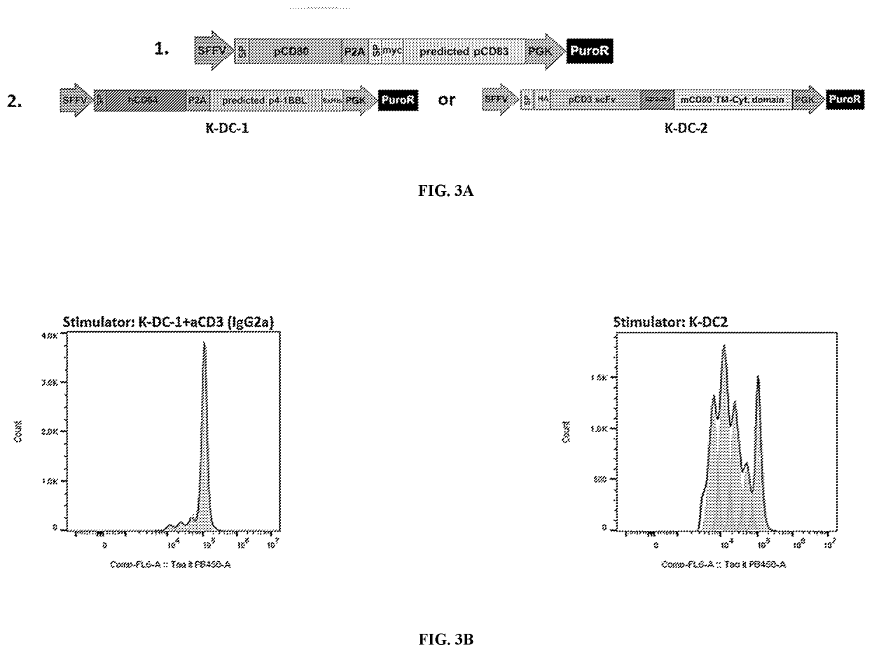

Artificial dendritic cells (aDC) for porcine cells were generated based on a human erythroleukemic cell line K562, which was stably transduced with the following vectors (FIG. 3A). Both constructs comprised a focus-forming virus (SFFV) promoter.
1. LV-SFFV-porcine CD80 (pCD80)-P2A-myc-porcine CD83 (p83) together with LV-SFFV-human CD64 (hCD64) hCD64-P2A-porcine 4-1BBL(6×His). The cells generated with these vectors are referred to as K-DC1. An exemplary nucleic acid encoding pCD80-P2A-myc-pCD83 is provided herein as SEQ ID NO: 3. An exemplary amino acid sequence comprising pCD80-P2A-myc-pCD83 is provided herein as SEQ ID NO: 20. An exemplary nucleic acid sequence encoding hCD64-P2A-p41BBL is provided herein as SEQ ID NO: 4. An exemplary amino acid sequence comprising hCD64-P2A-p41BBL is provided herein as SEQ ID NO: 22. Also provided is SEQ ID NO: 22 without a histidine tag.
2. LV-SFFV-pCD80-P2A-myc-pCD83 together with LV-SFFV-scFv-CD3-CD80™ (HA). The cells generated with these vectors are referred to as K-DC2. An exemplary sequence encoding pCD80-P2A-myc-pCD83 is provided herein as SEQ ID NO: 3. An exemplary amino acid sequence comprising pCD80-P2A-myc-pCD83 is provided herein as SEQ ID NO: 20. An exemplary sequence encoding HA-pCD3scFv-mCD80™ is provided herein as SEQ ID NO: 5.

### Sequences Used for Construction of Porcine aDC:

Sus scrofa (pig) 4-1BBL, TNFSF9 TNF superfamily member 9 Gene ID: 100736831. SEQ ID NO: 6 is an exemplary nucleic acid sequence encoding porcine 4-1BBL. An exemplary amino acid sequence comprising porcine 4-1BBL is provided herein as SEQ ID NO: 23.

Sus scrofa (pig) CD83 molecule, Gene ID: 100153365. SEQ ID NO: 7 is an exemplary nucleic acid sequence encoding porcine CD83. SEQ ID NO: 17 is an exemplary amino acid sequence comprising a porcine CD83.

Sus scrofa (pig) CD80 molecule, Gene ID: 397161 (Tadaki et al., Xenotransplantation 10)3: 252-258 (2003). SEQ ID NO:8 is an exemplary nucleic acid sequence encoding a porcine CD80. SEQ ID NO: 18 is an exemplary amino acid sequence comprising a porcine CD80.

Human FCGR1A or CD64, GenBank Accession No. NM_000566.4 CDS Gene ID: 2209. SEQ ID NO: 9 is an exemplary nucleic acid sequence encoding human CD64. SEQ ID NO:19 is an exemplary amino acid sequence comprising human CD64.

### Flow Cytometry Analysis:

A Cytoflex Flow Cytometer (BD Biosciences, Haryana India) was used to acquire immunofluorescence data and collected data were analyzed with FlowJo Software (BD Biosciences).

### Killing Assay and Intracellular IFNγ Staining

The functional activity of CAR-T cells was analyzed by their ability to kill CD19+ target cells. Cell line NALM-6 (CD19+) stably expressing F-Luciferase (NALM-6/F-Luc) was used as a specific target for CAR-T cells, while human cell line K562 (CD19−) expressing F-Luciferase was used as non-specific target in killing assay. NALM-6/F-Luc or K562/F-Luc were seeded in U-bottom 96-well plate at density 10,000 cells/well. CAR expressing or mock-transduced porcine T cells were added to the wells with target cells at different numbers to create an effector-to-target ratio from 0.25:1 to 10:1. Cells were incubated for a different period: 4-18 hours, for analysis cells were spin-down, re-suspended in PBS and transferred to 96-well plate with flat bottom and black walls. To each well, the BioGlo luciferase substrate was added and after 5 min of incubation chemiluminescence was read by Tecan spectrophotometer.

### Results

### Isolation and Activation of T Cells

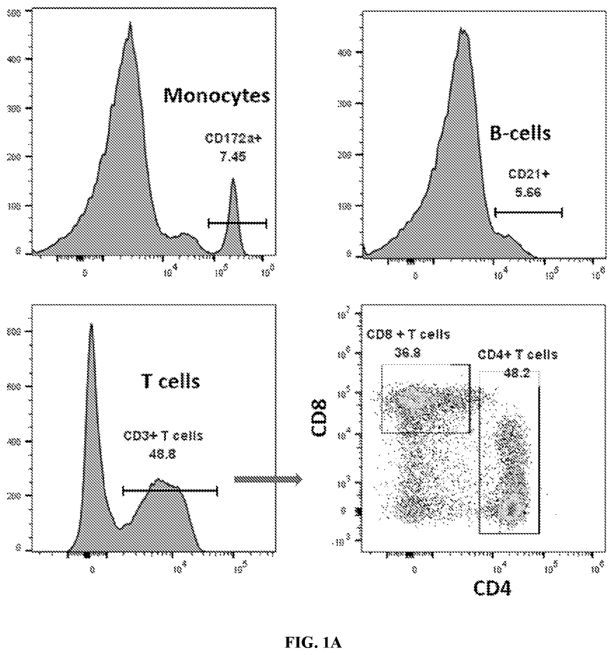

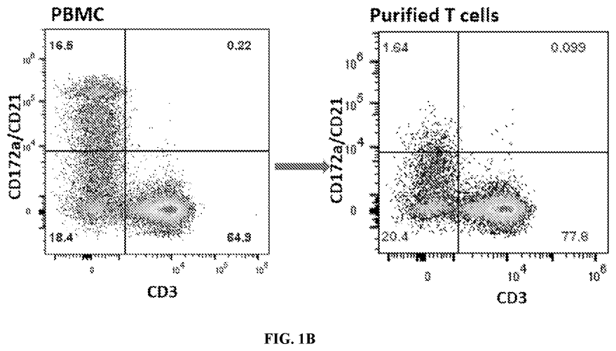

Porcine PBMC contains 45-55% of CD3⁺ T cells according to flow analysis (FIG. 1A). The removal of monocytes and B cells by negative selection increased the purify of T cells to 70-85% (FIG. 1B).

For lentiviral transduction, T cells should be in an activated/proliferative state. T-cell proliferation can be stimulated by many means including artificial and naturally-occurring chemicals such as phorbol esters or phytohemagglutinins. Among them, activation with CD3/CD28 antibodies is preferred since this mimics a natural stimulus and generates T cells with higher target affinity and prolonged life. For pigs, several clones of mouse anti-porcine CD3 antibodies available.

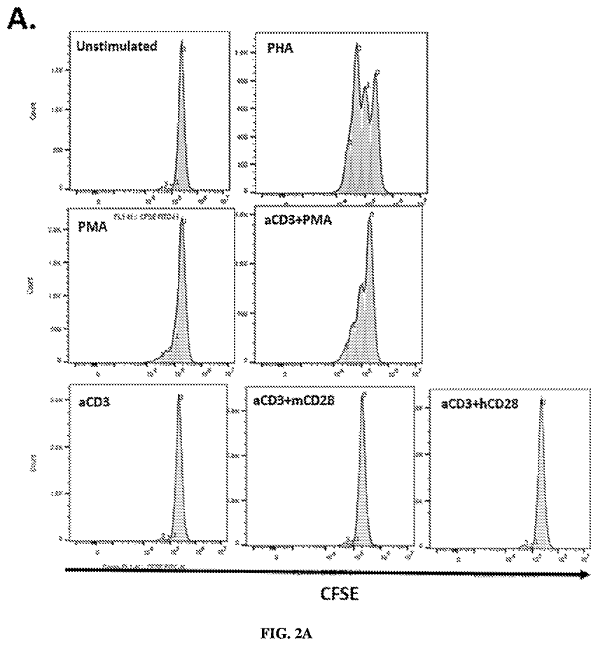

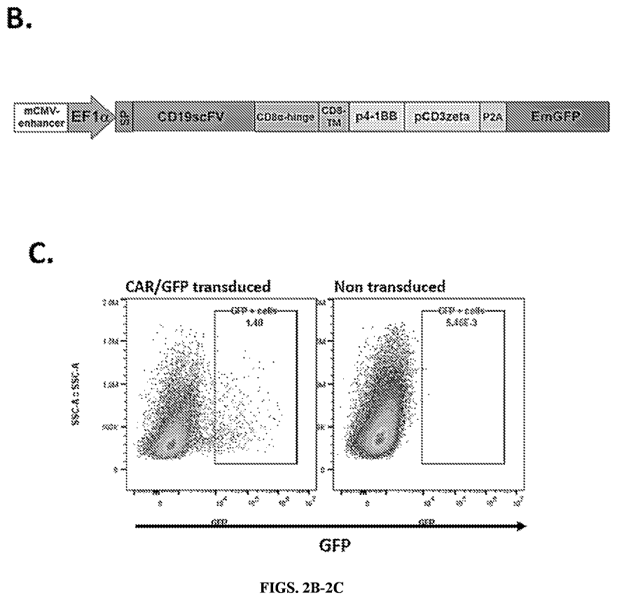

Whether anti-human and anti-mouse CD28 antibodies are cross-reactive with porcine T cells was tested. In addition, universal activators of T cells, such as phytohemagglutinin (PHA) and phorbol ester (PMA) were used. The activation state of T cells was judged by the level of T cell proliferation. CFSE labeled T cells were activated with 1) PMA or PHA alone, 2) PMA in combination with anti-porcine CD3 antibodies (PMA+aCD3), or 3) anti-porcine CD3 antibodies alone or in combination with human or mouse CD28 antibodies. The analysis of cell division 4 days later showed that PHA is the strongest stimulator followed by PMA in combination with anti-porcine CD3 antibodies. The anti-porcine CD3 antibodies alone or in combination with human or mouse CD28 antibodies did not activate T cells (FIG. 2A)

### Lentiviral Transduction

Cells activated by PHA, or by PMA and CD3 antibodies, were transduced with lentivirus encoding human CD19 specific chimeric antigen (CAR) and emGFP separated by a P2A peptide (FIG. 28, SEQ ID NO: 2). T cells were mixed with lentiviral vector supernatant in the presence of 4 μg/ml polybrene in Opti-MEM media and loaded into 24-multiwell plate. The plate was centrifuged at 800×g for 30 min at room temperature, and then placed in the cell culture incubator for an additional 3.5 hours. After four hours of transduction, an equal volume of RPMI supplemented with 10% FBS and 25 ng/ml pIL-2 was added to cells for overnight incubation. The next day, media was completely replaced with fresh 10% FBS-RPMI with 25 ng/ml pIL-2. Three days after transduction, the percentage of CAR⁺/GFP⁺ cells were determined by flow cytometry. Only 1% of PHA-stimulated T cells was GFP⁺, while PMA plus CD3 antibody stimulated T cells were not transduced by LV (FIG. 2C). Moreover, the activation by PHA induced activation-induced apoptotic death of T cells and, as a result, T cells were unable to expand. The poor transduction of T cells indicated that the conditions for T cell activation and culturing should be optimized.

To optimize activation and culturing, several approaches were pursued. Artificial dendritic cells were created; the role of media and cytokines in maintenance of CAR-T cells was investigated; different transduction enhancers were tested; and the role of the promotor on transduction efficiency was examined.

### Generation of Artificial Dendritic Cells

Since the combination of anti-porcine CD3 and CD28 antibodies is unavailable for stimulation of T cells, artificial dendritic cells that could provide natural stimulus and potentially generate T cells with higher target affinity and prolong life were made. Artificial dendritic cells (ADC) can be created based on 3T3 or K562 cell lines stably transfected with molecules necessary to deliver all appropriate signals for T cells activation, expansion, and function (Latouche and Sadelain, Nature Biotechnology 18(4): 405-409 (2000). To serve as a backbone of cell-based and gene-engineered ADC, several requirements must be satisfied. First, the induction of allspecific T cells by ADC should be absent or minimal. Therefore, the backbone cells should not express any HLA class I or II molecules. Second, they should be easily gene-manipulated. Third, a track record of previously using the backbone cell line safely in human beings would be preferable. Since K562 cells satisfy all three of these requirements, they have been widely utilized as a backbone cell line for a series of ADC by many investigators (Butler and Hirano, Immunological Reviews 257(1): 191-209 (2014)).

Two versions of ADCs were created. Both were equipped with a porcine CD80 molecule, which provided the same co-stimulatory signal as CD28 antibodies. In addition, co-stimulatory molecule CD83 also was included (FIG. 3). For the signal of CD3 antibodies, two separate constructs were used (FIG. 3A). The first construct included human CD64 receptor, which is a high affinity receptor for IgG2a antibodies. CD4, on the surface of ADCs, binds externally added anti-porcine CD3 IgG2a antibodies (FIG. 3A). Alternatively, instead of binding to CD64, membrane-bound scFv CD3 antibodies can be directly expressed on the surface of ADC (Liao et al., Gene Therapy 7(4): 339-347 (2000). Therefore, the second construct encoded the sequence of membrane-bound anti-porcine scFv CD3 antibodies (FIG. 3A), which was based on a publicly available published sequence (Wang et al. Bioconjugate chemistry 22(10): 2014-2020 (2011). It is important to note that CD83 and 4-1BBL (CD137L) were added to constructs as important co-stimulatory molecules to support long term growth of T cells (Takahashi et al. The Journal of Immunology 162 (9): 5037-5040 (1999).

In the first round of transduction, K562 cells, i.e., the artificial dendritic cells described above, were infected with a lentivirus encoding porcine CD80 and CD83 separated by a P2A peptide (FIG. 3A). After transduction, cells were FACS sorted based on CD80 expression on the cell surface, and cells were expanded in the presence of selective antibiotic puromycin. In the second round, K562 cells stably expressing CD80 and CD83 were transduced either with lentiviral-encoded membrane-bound scCD3 antibodies (K-DC2) or with LV encoded CD64 and 4-1BBL (K-DC1) (FIG. 3A (2)).

Transduced cells were selected by FACS and expanded in the presence of selective antibiotic puromycin. Before using them in experiments with T cells, K-DC1 and K-DC2 were treated with mitomycin C (0.5 mg/ml, 1.5 h) to prevent their division. In addition, K-DC1 which expresses CD64, a high affinity receptor for IgG2a antibodies were incubated with anti-porcine CD3 antibodies (WSU, 8E6-8C8, IgG2a) which results in the presence of CD3 antibodies on the surface of K-DC1. T cells were labeled with carboxyfluorescein diacetate succinimidyl ester (CFSE) and co-incubated with K-DC1 or K-DC2 at a 5:1 ratio (T cells-to-K-DC). Analysis of T cell proliferation after 4 days of co-incubation with K-DC showed that K-DC2 are superior in induction of T cells proliferation (FIG. 3B), while K-DC1 were less efficient and induced only moderate proliferation (FIG. 3B). In subsequent experiments K-DC2 were used.

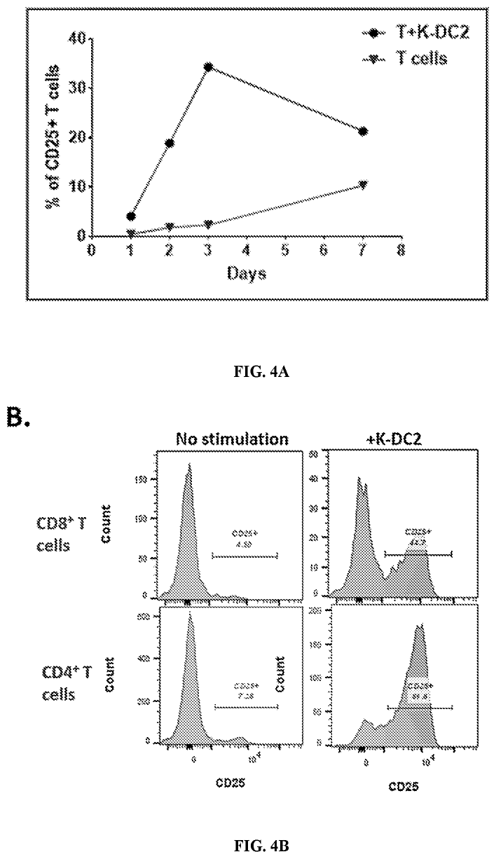

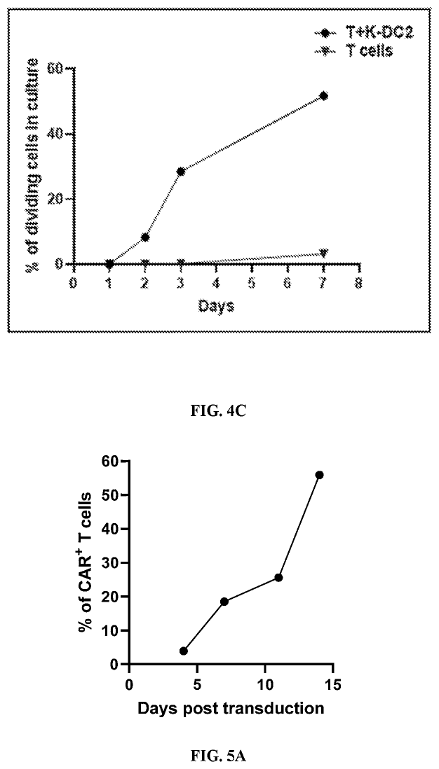

The proper activation of porcine T cells by K-DC2 was also confirmed by analyzing the levels of CD25 on the surface of T cells. Upon stimulation, the expression of CD25 dramatically increased and reached maximum at day 3 (FIG. 4A, B). This single stimulation with K-DC2 cells induced prolonged proliferation of T cells as confirmed by dilution assay (FIG. 4C)

### Optimization of Media Formulation to Improve the Effectiveness of CAR-T Cell Production and Functional Activity

It is generally accepted that the success in CAR T cell production is highly depends on the optimal cell culture methods during the activation and expansion of T cells ex vivo, as well as during the infection with CAR. It appears that the differentiation state of CAR-T cells dramatically affects their survival and killing ability. However, which differentiation state is most beneficial for adoptive transfer is not clear and still the area of active research.

Accumulated data suggest that young phenotype or stem/memory T cells have improved function and survival (Gargett et al. Cytotherapy 17(4): 487-495 (2015). In addition, the ability to modulate the differentiation state of CAR-T cells population by manipulation in media formulation is very attractive strategy due to easiness and cost efficiency.

The choice of proper cytokines is very important and can reduce the activation-induced T cell death. Interleukin-2 (IL-2) and Interleukin-21 (IL-21) are two closely related families of signaling molecules that arose from gene duplication. Both cytokines are pleiotropic molecules that support the maturation and differentiation of CD8+ effector T cells. Both cytokines have been widely used in adoptive immunotherapy as growth factors during ex vivo expansion of T cells. Another frequently used cytokine for T cell expansion is IL-7, but IL-7 is highly species specific and porcine IL-7 is not commercially available.

In the studies described herein, human IL-2 (Biolegend, San Diego, CA) was compared with porcine IL-2 (Biolegend) alone or in combination with porcine IL-21 (Kingfisher Biotech, St. Paul, MN), since hIL-2 is cross-species reactive and recognized by porcine cells. T cells were cultured in 10% FBS-RPMI supplemented with different cytokines before and after transduction. Cell numbers and transduction percentage were measured. Next, cytokines were used:
25 ng/ml hIL-2
25 ng/ml pIL-2
25 ng/ml pIL-2 together with 10 ng/ml rpIL-21
30 ng/ml p-IL-21.

It was found that human IL-2 (hIL-2) supports porcine T cell proliferation at the same level or better than porcine IL-2 (pIL-2). Porcine IL-21 alone (30 ng/ml pIL-21) doesn't support T cell proliferation. However, the combination of hIL-2 and pIL-21 supported T cell expansion better than porcine IL-2 or human IL-2. Therefore, this combination of cytokines (i.e., hIL-2 and pIL-21) can be used to maintain porcine T-cells.

### Optimization of Transduction

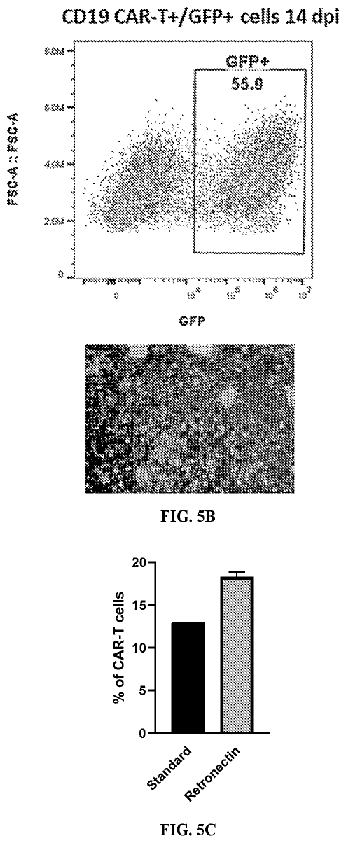

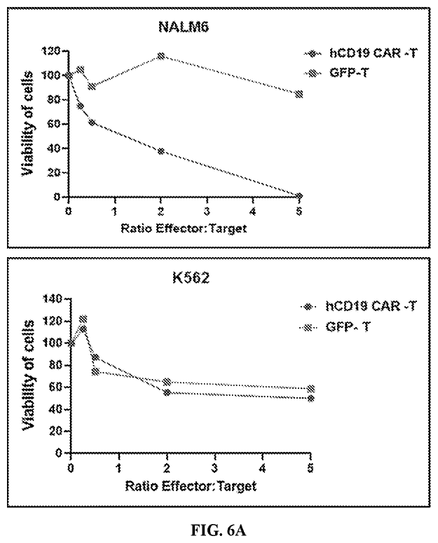

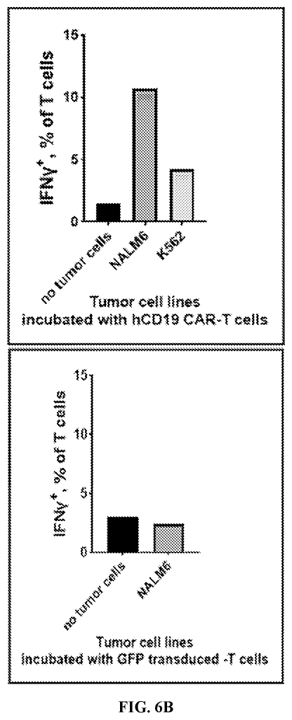

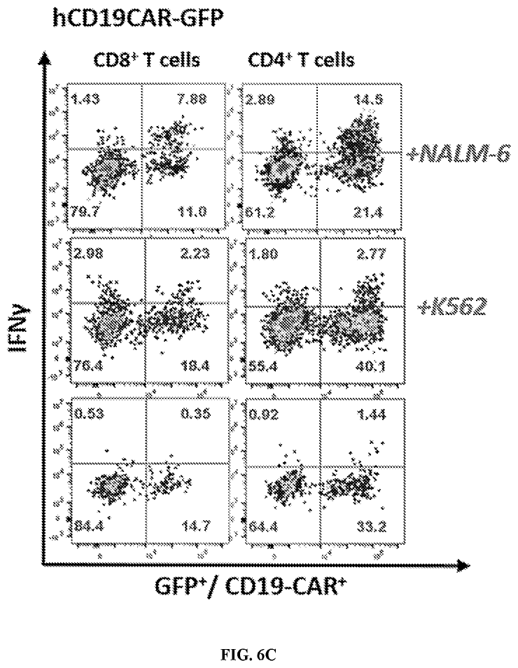

Three transduction enhancers were compared: polybrene (Sigma), retronectin (Takara) and vectofusin-1 (Miltenyi). All enhancers were tested on T cells activated with K-DC2 for 3 days with LV-CD19CAR-GFP at MOI-10. For retronectin enhanced transduction, retronectin pre-coated plates w % ere used. The level of transduction was estimated by GFP expression on day 3, post infection. In the presence of polybrene, 4.6% of T cells were GFP+; in the presence of vectofusin-13.7% of T cells were GFP+; and no GFP+ cells were detected in retronectin-transduced cells. Polybrene was the best enhancer of transduction and the most-cost-effective. However, the activation-induced death of T cells was lower in retronectin-coated plates compared to standard plates. Retronectin can improve retroviral transduction and enhance the proliferation of T lymphocytes. Moreover, T cells expanded on retronectin-coated plates can contain a high proportion of less-differentiated T cells. Hence, in the next experiments polybrene was chosen as an enhancer of lentivirus-mediated gene transduction, but T cells were cultured on retronectin-coated plates to improve their expansion and phenotype. As a result, the transduction with polybrene as enhancer, on retronectin-coated plates, consistently gave better results compared to polybrene alone (FIG. 5A).

Although the initial level of transduction with CAR was around 5-10%, depending on PBMC source, as this can vary from pig to pig, during the period of T cell expansion, after transduction, the levels of CAR+ T cells in culture increased up to 55% (FIG. 5B, C). This was observed only in T cells comprising a CAR-lentivirus comprising an eF1α promoter (map shown in FIG. 2B), but not for a CAR lentivirus comprising a SFFV promoter.

The modifications described herein allowed generation of a pipeline for porcine CAR T cell production. Briefly, PBMC-derived porcine T cells, at 75-85% purity were used for the generation of human-specific CD19 CAR T cells. T cell fractions were enriched by removing CD172a (monocytes), CD21 (B cells) and CD16 (NK and neutrophils) positive cells from PBMC by using corresponding antibodies and magnetic beads. After enrichment, T cells were plated in retronectin-coated dishes in 10% FBS-AIM-V media supplemented with 25 ng/ml hIL2 and 10 ng/ml pIL21 and stimulated with artificial dendritic cells at a ratio of 5 to 1. On day 3 or 4, at the peak of activation (determined by the expression of CD25 on cell surface) T cells were transduced with a lentiviral vector encoding CD19 CAR and GFP at a multiplicity of infection ranging from 3 to 10 using a spinoculation technique (plate with cells and LV was centrifuged at 800×g for 2 hrs), in serum-free Aim-V media supplemented with 8 μg/ml polybrene (PB) and in retronectin-coated plates. For mock transduction, a lentiviral vector encoding only GFP was used. The day after transduction, media was replaced with 10% FBS-AIM-V, supplemented with 50 ng/ml hIL2 and 10 ng/ml pIL21, and cells were cultured in the same retronectin-coated plates until expansion. During the next 10-14 days transduced T cells were expanded, and during expansion transferred to standard, non-coated with retronectin cells suspension flasks. At the end of expansion, cells were used for experiments or frozen in CryoStor media at density 10⁷ cells/ml.

### Cancer Therapy

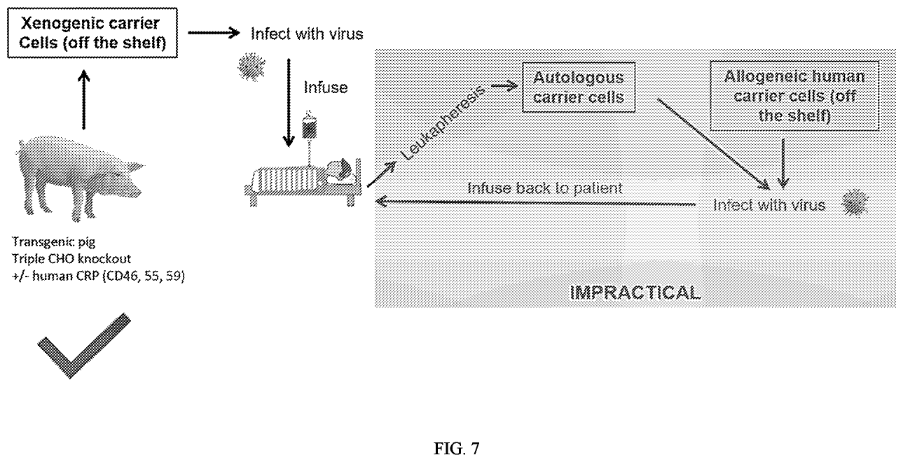

As shown in FIG. 7, there are advantages to using transgenic ungulate cells as carriers for oncolytic viruses. It can often be impractical to obtain autologous carrier cells from a subject or allogeneic cells from a different subject or commercially available source, infect these autologous or allogeneic cells with a virus, and then infuse them back into the subject. The cells and methods provided herein provide readily available cells from a transgenic ungulate that is modified to reduce rejection of a cell or tissue derived from the transgenic animal when transplanted into a human host. These cells can be infected with an oncolytic virus and subsequently infused into the subject. These cells include, for example, and not to be limiting, T cells, NK cells, B cells, monocytes, and macrophages.

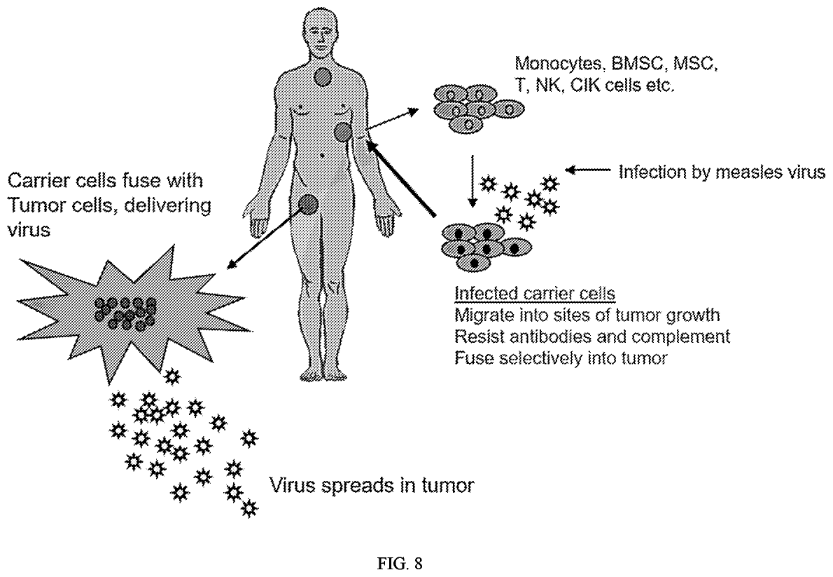

FIG. 8 is an exemplary schematic for circumventing antiviral antibodies using transgenic ungulate carrier cells as delivery vehicles for an oncolytic measles virus. Monocytes, bone marrow derived stem cells, mesenchymal stem cells, T cells NK cells, unsorted PBMCs or cytokine induced killer cells derived from a transgenic ungulate described herein can be infected with an oncolytic measles virus and subsequently administered to a subject that has cancer. The infected carrier cells will migrate to sites of tumor growth, resist antibodies and complement and fuse selectively with tumor cells, to deliver the virus to the tumor cells.

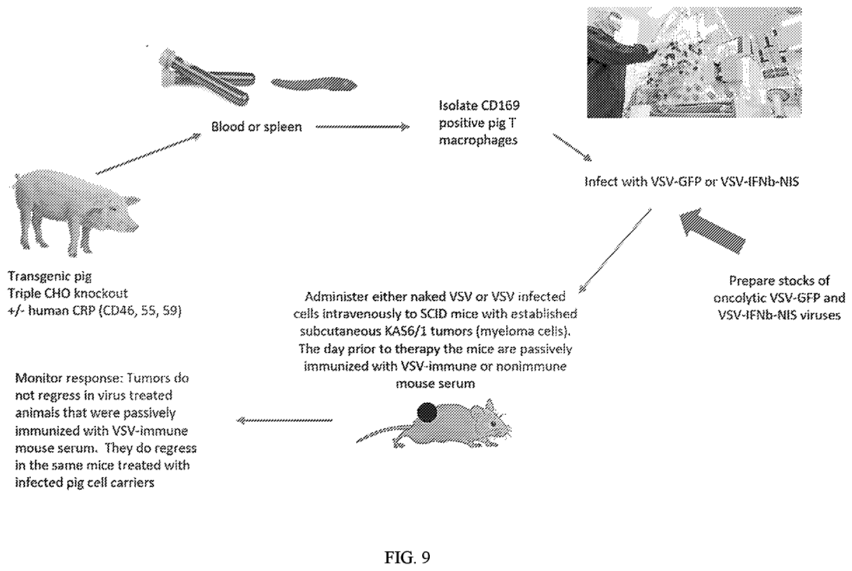

FIG. 9 is an exemplary schematic for circumventing VSV antibodies and treating a human cancer using VSV-infected CAR-expressing pig T cells engineered to evade hyperacute rejection in a human. As shown in FIG. 3, transgenic pig CD169+ macrophages are obtained from the blood or spleen of a triple CHO knockout pig (+/− heterologous expression of CD46, CD55 and/or CD59). The CD169+ macrophages are then infected with VSV-GFP or VSV-IFN beta-NIS. After infection, naked VSV or VSV infected cells are administered intravenously to SCID mice with established subcutaneous KAS6/1 tumors (myeloma cells). The day prior to therapy, the mice are passively immunized with VSV-immune or nonimmune mouse serum. After administration, the response is monitored in the mice. Tumors should not regress in virus treated animals that are passively immunized with VSV-immune mouse serum. They should regress in the same mice treated with infected pig cell carriers.

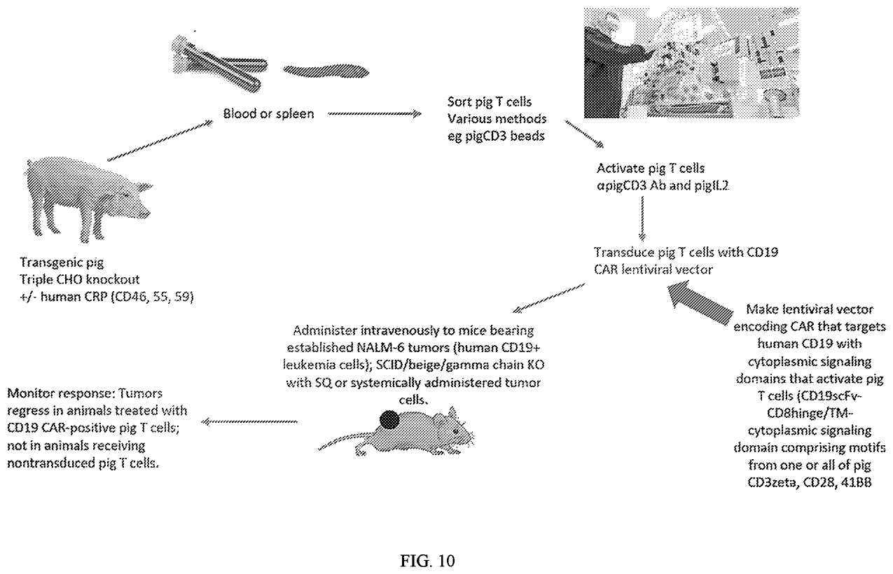

FIG. 10 is an exemplary schematic for the treatment of a human cancer with CAR-expressing pig T cells engineered to evade hyperacute rejection in a human. As shown in FIG. 4, transgenic pig T cells are obtained from the blood or spleen of a triple CHO knockout pig (+/− heterologous expression of CD46, CD55 and/or CD59). The pig T cells are sorted and subsequently activated with an anti-CD3 antibody and pig IL-2. The pig T cells are then transduced with a lentiviral vector encoding a CD19 CAR. The lentiviral vector encoding CAR targets human CD19 with cytoplasmic signaling domains that activate pig T cells. The exemplary construct (SEQ ID NO: 1 or SEQ ID NO: 2) encodes a CAR comprising a CD19scFv, a CD8 hinge region, and a transmembrane-cytoplasmic signaling domain comprising motifs from one or all of pig CD3zeta, CD28, and 4-1BB, as shown in FIG. 12. After transduction, the cells are intravenously administered to mice bearing established NALM-6 tumors (human CD19+ leukemia cells), SCID/beige/gamma chain KO with subcutaneously (SQ) or systemically administered tumor cells. After administration, the response is monitored in the mice. Tumors should regress in animals treated with CD19 CAR+ pig T cells as compared to animals receiving nontransduced pig T cells.

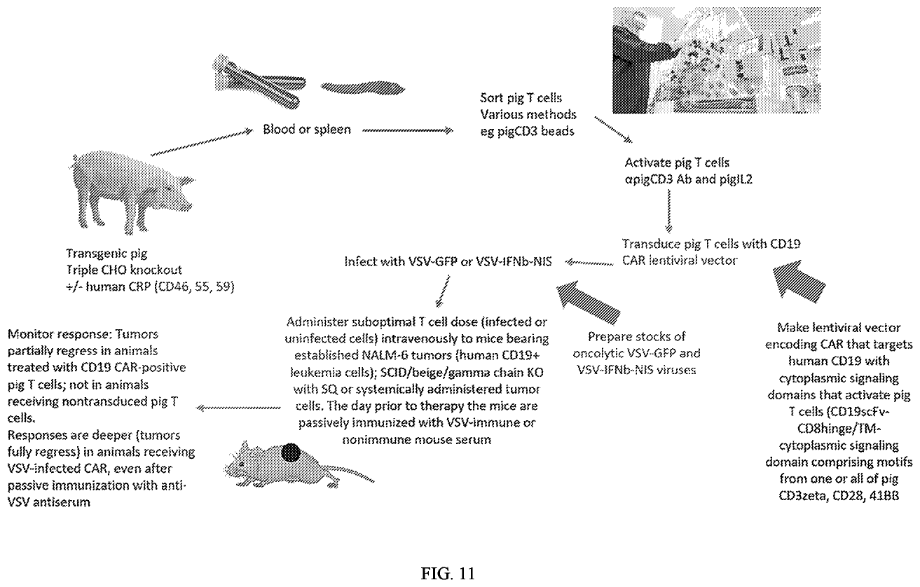

FIG. 11 is an exemplary schematic for the treatment of a human cancer using VSV-infected, CAR-expressing pig T cells engineered to evade hyperacute rejection in a human. As shown in FIG. 5, transgenic pig T cells are obtained from the blood or spleen of a triple CHO knockout pig (+/− heterologous expression of CD46, CD55 and/or CD59). The pig T cells are sorted and subsequently activated using with an anti-CD3 antibody and pig IL-2. The pig T cells are then transduced with a lentiviral vector encoding a CD19 CAR. The lentiviral vector encoding CAR targets human CD19 with cytoplasmic signaling domains that activate pig T cells (CD19scFv-CD8 hinge/TM-cytoplasmic signaling domain comprising motifs from one or all of pig CD3zeta, CD28, and 4-1BB). After transduction, the CAR+ cells are infected with VSV-GFP or VSV-IFN beta-NIS.

After infection, a suboptimal T cell dose (infected or uninfected cells) is administered intravenously to mice bearing established NALM-6 tumors (human CD19+ leukemia cells); SCID/beige/gamma chain KO with SQ, or systemically administered tumor cells. The day prior to therapy, the mice are passively immunized with VSV-immune or nonimmune mouse serum. After administration, the response is monitored in the mice. Tumors should partially regress in animals treated with CD19 CAR-positive pig T cells, but not in animals receiving nontransduced pig T cells. Responses should be deeper (tumors fully regress) in animals receiving VSV-infected CAR, even after passive immunization with anti-VSV antiserum.

FIG. 12 is an exemplary schematic of a construct (SEQ ID NO: 1) that encodes a CAR comprising a CD19scFv, a CD8 hinge region, and a transmembrane-cytoplasmic signaling domain comprising motifs from one or all of pig CD3zeta, CD28, and 4-1n.

### Sequences

### Seq Id No: 1

Nucleic acid sequence comprising (a) a nucleic acid sequence encoding an anti-CD19 scFv, (b) a nucleic acid encoding a CD8α hinge, (c) a CD8 transmembrane domain, (c) a porcine, cytoplasmic 41BB signaling domain, and (d) a cytoplasmic porcine CD3 zeta signaling domain

SEQ ID NO: 2
tctagaatggccttaccagtgaccgccttgctcctgccgctggccttgctgctccacgccgccaggccggacatccagat
gacacagactacatcctccctgtctgcctctctgggagacagagtcaccatcagttgcagggcaagtcaggacattagta
aatatttaaattggtatcagcagaaaccagatggaactgttaaactcctgatctaccatacatcaagattacactcagga
gtcccatcaaggttcagtggcagtgggtctggaacagattattctctcaccattagcaacctggagcaagaagatattgc
cacttacttttgccaacagggtaatacgcttccgtacacgttcggaggggggaccaagctggagatcacaggtggcggtg
gctcgggcggtggtgggtcgggtggcggcggatctgaggtgaaactgcaggagtcaggacctggcctggtggcgccctca
cagagcctgtccgtcacatgcactgtctcaggggtctcattacccgactatggtgtaagctggattcgccagcctccacg
aaagggtctggagtggctgggagtaatatggggtagtgaaaccacatactataattcagctctcaaatccagactgacca
tcatcaaggacaactccaagagccaagttttcttaaaaatgaacagtctgcaaactgatgacacagccatttactactgt
gccaaacattattactacggtggtagctatgctatggactactggggccaaggaacctcagtcaccgtctcctcaaccac
gacgccagcgccgcgaccaccaacaccggcgcccaccatcgcgtcgcagcccctgtccctgcgcccagaggcgtgccggc
cagcggcggggggcgcagtgcacacgagggggctggacttcgcctgtgatatctacatctgggcgcccttggccgggact
tgtggggtccttctcctgtcactggttatcaccctttactgcaaacggggcagaaagaaactcctgtatatattcaaaca
accatttatgagaccagtacaaactactcaagaggaagatggctgtagctgccgatttccagaagaagaagaaggaggat
gtgaactgagagtgaagttcagcaggagcgcagacgcccccgcgtacaagcagggccagaaccagctctataacgagctc
aatctaggacgaagagaggagtacgatgttttggacaagagacgtggccgggaccctgagatggggggaaagccgagaag
gaagaaccctcaggaaggcctgtacaatgaactgcagaaagataagatggcggaggcctacagtgagattgggatgaaag
gcgagcgccggaggggcaaggggcacgatggcctttaccagggtctcagtacagccaccaaggacacctacgacgccctt
cacatgcaggccctgccccctcgctaagtcgac

Nucleic acid sequence comprising (a) a nucleic acid sequence encoding an anti-CD19 scFv, (b) a nucleic acid encoding a CD8α hinge, (c) a CD8 transmembrane domain, (c) a porcine, cytoplasmic 41BB signaling domain, and (d) a cytoplasmic porcine CD3 zeta signaling domain

ATGGCCTTACCAGTGACCGCCTTGCTCCTGCCGCTGGCCTTGCTGCTCCACGCCG
CCAGGCCGGACATCCAGATGACACAGACTACATCCTCCCTGTCTGCCTCTCTGGG
AGACAGAGTCACCATCAGTTGCAGGGCAAGTCAGGACATTAGTAAATATTTAAA
TTGGTATCAGCAGAAACCAGATGGAACTGTTAAACTCCTGATCTACCATACATCA
AGATTACACTCAGGAGTCCCATCAAGGTTCAGTGGCAGTGGGTCTGGAACAGAT
TATTCTCTCACCATTAGCAACCTGGAGCAAGAAGATATTGCCACTTACTTTTGCC
AACAGGGTAATACGCTTCCGTACACGTTCGGAGGGGGGACCAAGCTGGAGATCA
CAGGTGGCGGTGGCTCGGGCGGTGGTGGGTCGGGTGGCGGCGGATCTGAGGTGA
AACTGCAGGAGTCAGGACCTGGCCTGGTGGCGCCCTCACAGAGCCTGTCCGTCA
CATGCACTGTCTCAGGGGTCTCATTACCCGACTATGGTGTAAGCTGGATTCGCCA
GCCTCCACGAAAGGGTCTGGAGTGGCTGGGAGTAATATGGGGTAGTGAAACCAC
ATACTATAATTCAGCTCTCAAATCCAGACTGACCATCATCAAGGACAACTCCAAG
AGCCAAGTTTTCTTAAAAATGAACAGTCTGCAAACTGATGACACAGCCATTTACT
ACTGTGCCAAACATTATTACTACGGTGGTAGCTATGCTATGGACTACTGGGGCCA
AGGAACCTCAGTCACCGTCTCCTCAACTACGACTCCAGCACCACGACCACCAAC
ACCAGCGCCCACCATTGCAAGCCAGCCACTTTCACTGCGCCCAGAAGCGTGTAG
ACCAGCTGCTGGAGGAGCAGTGCATACGAGAGGGCTGGACTTCGCCTGTGATAT
CTACATCTGGGCGCCCTTGGCCGGGACTTGTGGGGTCCTTCTCCTGTCACTGGTT
ATCACCCTTTACTGCCAACAGGGCAGGAAGAAACTCCTGTATATAGTCAAACAA
CCGTTTCTGAAGCCAGCGCAAACGGTCCAAGAGGAAGATGCCTGCAGCTGCCGG
TTTCCAGAAGAAGAGGAAGGCGAGTGTGAACTAAGAGTGAAGTTCAGCAGGAG
CGCAGACGCCCCCGCGTACCAGCAGGGCCAGAACCAGCTCTATAACGAGCTCAA
TCTAGGACGAAGAGAGGAGTACGATGTTTTGGACAAGAGACGTGGCCGGGACCC
TGAGATGGGGGGAAAGCCGAGGAGGAAGAACCCTCAGGAAGGCCTGTACAATG
AACTGCAGAAAGATAAGATGGCGGAGGCCTACAGTGAGATTGGGATGAAAGGC
GAGCGCCGGAGGGGCAAGGGGCACGATGGCCTTTACCAGGGTCTCAGTACAGCC
ACCAAGGACACCTACGACGCCCTTCACATGCAGGCCCTGCCCCCTCGC
pCD80-P2A-myc-pCD83:
SEQ ID NO: 3
ATGTGTCACACACTGAAGTGGGGAACACCATTACCCAAGCTCTTTCAGCTCTTGG
TGCTGGTTGGTCTTTTTGACTTCTGTTCAGGCATCGTTCAGGTGACCAAAACAGTG
AAAGAAATAGCAGTGCTATCCTGTGATTACAACATATCCACTGAAGAACTGACT
AGAGTCCGAATATACTGGCAAAAGGATAATGAAATGGTGCTGGCTGTCATGTCT
GGAAAAGTGAAGGTGTGGCCCAAGTATGAGAACCGCACCTTCACTGATGTCACC
AATAACCTCTGCATTGTGATCCTGGCTCTGCGCCTGTCAGACAATGGCACCTACA
CCTGTGTTGTTCAGAAGCGGGAGAGAGGGTCTTATAAGCTGGAGCACCTGACTTC
GGTGAAGTTAATGGTCAAAGCTGACTTTCCTGTGCCTAGTATTACTGCCCTTGGA
AATCCATCTCCTAACATCAAAAGGATAAGGTGCTCAACCTCTGGAGGTTTTCCAG
AGCCTCACCTCTCCTGGTTGGAAAATGGAGAAGAATTAAATGCTACCAACACGA
TGCTTTCCCAAGATCCTGAAACTGAGCTCTACATGATTAGCAGTGAACTGGATTT
CAATGTGACAGGCAACCACAGCTTCATGTGTCTTGTCAAGTATGGAGGCTTAACA
GTGTCACAGACCTTCAACTGGCAAAAATCAGCTAAACGAGAAACCTCTTCTGCTA
ATCAGTTCCTGCTCTTCACCGTTATTATATCAGCCTCAGCATGTGGGATTGCAATA
ATACTAATATACCGGCTCTGCAGATCTGCTGCTCCAAGACAGAGAAGGAGGAGG
AATATGGAGAGCATGGAAATGGAAAGGATCTCCCCAATCTACCCAGGATCTGTG
GAAGGACCAGGCACCGTCGGAAGCGGAGCTACTAACTTCAGCCTGCTGAAGCAG
GCTGGAGACGTGGAAGAGAACCCTGGACCTAGATCCATGGCCACAACCATGTCG
CGCGGCCTCCAGCTTCTGCTCCTGAGCTGCGCCTACAGCCTGGCTCCCGCGGAAC
AAAAACTTATTAGCGAAGAAGATCTTGTGCTGGAGGTGAAAGTGGTCTGTTCGG
AGGCTGTGGACTTGCCCTGCCCCGTCCCCTGGGACCCGCAGGTCCGCTACACGGT
GTCCTGGGCCAAGCTGTCCGGCGGCCGTGCAGAGAGCCTGGAGGTGCCCGGGGA
AGAGCTGCGGTCCGCTCTCCAGAATGGCTCTTCGGGGGCCCCCAGTGGGAGGCT
GTATTCTCTGAAGATCCAGAACACTACCAGCTGCAACTCGGGGGCCTACCGCTGC
ACTCTGGAGGACCCGGAGACCCAGAGAAACCTCAGCGGCACCGTGATCTTGAAA
GTGACAGGATGCTCTAAGGGACGCAAAGAAGATACTTTTAAGAAATACAGAGCT
GAGATTGTCCTGCTGTTGGCTCTGGTCACTTTCTACTTAACACTCATCATTTTCAC
CTGCAAGTTTGCACGACAGCAGAGTATTTTTCCAGATTTTTCTAAACCGGTCGTA
GAACATGCTTTTCTCCCCGTCACCTCCCCAAATAAGCCCTTGGAGCCAGTGACTC
TTCACAAGACAGAGCTGGTATAA
hCD64-P2A-p41BBL
SEQ ID NO: 4
ATGTGGTTCTTGACAACTCTGCTCCTTTGGGTTCCAGTTGATGGGCAAGTGGACA
CCACAAAGGCAGTGATCACTTTGCAGCCTCCATGGGTCAGCGTGTTCCAAGAGG
AAACCGTAACCTTGCACTGTGAGGTGCTCCATCTGCCTGGGAGCAGCTCTACACA
GTGGTTTCTCAATGGCACAGCCACTCAGACCTCGACCCCCAGCTACAGAATCACC
TCTGCCAGTGTCAATGACAGTGGTGAATACAGGTGCCAGAGAGGTCTCTCAGGG
CGAAGTGACCCCATACAGCTGGAAATCCACAGAGGCTGGCTACTACTGCAGGTC
TCCAGCAGAGTCTTCACGGAAGGAGAACCTCTGGCCTTGAGGTGTCATGCGTGG
AAGGATAAGCTGGTGTACAATGTGCTTTACTATCGAAATGGCAAAGCCTTTAAGT
TTTTCCACTGGAATTCTAACCTCACCATTCTGAAAACCAACATAAGTCACAATGG
CACCTACCATTGCTCAGGCATGGGAAAGCATCGCTACACATCAGCAGGAATATC
TGTCACTGTGAAAGAGCTATTTCCAGCTCCAGTGCTGAATGCATCTGTGACATCC
CCACTCCTGGAGGGGAATCTGGTCACCCTGAGCTGTGAAACAAAGTTGCTCTTGC
AGAGGCCTGGTTTGCAGCTTTACTTCTCCTTCTACATGGGCAGCAAGACCCTGCG
AGGCAGGAACACATCCTCTGAATACCAAATACTAACTGCTAGAAGAGAAGACTC
TGGGTTATACTGGTGCGAGGCTGCCACAGAGGATGGAAATGTCCTTAAGCGCAG
CCCTGAGTTGGAGCTTCAAGTGCTTGGCCTCCAGTTACCAACTCCTGTCTGGTTTC
ATGTCCTTTTCTATCTGGCAGTGGGAATAATGTTTTTAGTGAACACTGTTCTCTGG
GTGACAATACGTAAAGAACTGAAAAGAAAGAAAAAGTGGGATTTAGAAATCTCT
TTGGATTCTGGTCATGAGAAGAAGGTAATTTCCAGCCTTCAAGAAGACAGACATT
TAGAAGAAGAGCTGAAATGTCAGGAACAAAAAGAAGAACAGCTGCAGGAAGGG
GTGCACCGGAAGGAGCCCCAGGGGGCCACGGTCGGAAGCGGAGCTACTAACTTC
AGCCTGCTGAAGCAGGCTGGAGACGTGGAAGAGAACCCTGGACCTAGATCCATG
GCCACAACCATGAGCTCCAGCTCCGACGTCGACCAGGACCCCGAGGCCCAGAGG
CCTCCCGCGCCCCGAGGCCGAGCTTGCAGCCCCTTGCCCTGGGCCTTGAGCGCCG
CGCTGCTAGTGTTCGCAGCCGCCAGCGCCGTCTGCCTGGTGCGCTCCTGGACCGC
GCCCGGGGCCCCCGCCGCGCCCGGTCCCGCGCCCAGCCCGCGACTGCCAGAGGG
TCCGGAGTTGACGTCCGACGCCCTGGCCGGCTTCCCAGACTATCCACAGGGCATG
TTCCAGCAGGATGTTTTCGCGCAGCTGGTGGTGGCGGAGGGTGAAAAGCTGACC
GAAGGGCCCCTGCACTGGTGCAGCGAGCCAGGGATGAAAGGTGTGACTCTGACG
CCAGGTGTGAGATATGACAGGGACACACATGAGCTGGTGGTGGTCAAGGCTGGA
ATCTACTATGTGTTCTTGCACTTGGCACTGAAGCGCGTGGTGGCCGTCAACAGCA
ACAGCAACAGCAACATCAGCTCCGGCTCCGTCTCCGTTGCCCTGCACCTGCAGCC
TTCCCACGCTGAGGCCACCGCCTTGGCCTTGACCTTGGACCTGCCACCCCCATCC
TCAGGGAACTCAGCCGCTGGTTTCCGGGGTGGCCTGCTGCACCTGGGTGCTGGAC
AGCGCCTGGGTGTCCACCTGCACCCTGAACTCCGCAAGCCCCTCACCTGGCAGCT
CTTGGACACCACGGCCTTGGGCCTCTTCCGTGTGGCCACCAGAGCCCCTGCTGTA
CTCCCTTCACACCAGCTCTCACACCATCATCACCATCACTAA
HA-pCD3scFv-mCD80TM
SEQ ID NO: 5
ATGCGCACCCCCGCCCAGTTCCTGGGCATCCTGCTGCTGTGGTTCCCCGGCATCA
AGTGCTACCCGTATGATGTTCCGGATTACGCTGAAACAACTGTGACCCAGTCTCC
AGCATCCCTGTCCATGGCTATAGGAGAACAAGTCACCATCAGATGCATAACCAG
CACTGATATTGATGATGATATGAACTGGTACCAGCAGAGGCCAGGGAAACCTCC
TAAGCTCCTTATTTCAGAAGGCAATACTCTTCGTCCTGGAGTCCCATCCCGATTCT
CCAGCAGTGGCTATGGTACAGATTTTGTTTTTACAATTGAAAACATGCTCTCAGA
GGATGTTGCAGATTACTACTGTTTACAAAGTGATAACTTGCCGCTCACGTTCGGT
GCTGGGACCAAGCTGGAGCTGAAAGGTGGAGGCGGTTCAGGCGGAGGTGGCTCT
GGCGGTGGCGGATCGGAAGTGAAGCTGGTGGAGTCTGGGGGAGGCTTAGTGAAG
CCTGGAGGGTCCCTGAAACTCTCCTGTGCAGCCTCTGGATTCACTTTCAGTAGCT
ATACCATGTCTTGGGTTCGCCAGACTCCGGAGAAGAGGCTGGAGTGGGTCGCAT
ACATTAGTAGTGGTGGTAGTAATTCCTACTATTCAGACAGTGTGAAGGGCCGATT
CACCATCTCCAGAGACAATGCCAGGAACACCCTGTACCTGCAAATGAGCAGTCT
GAGGTCTGAGGACACGGCCATGTATTACTGTGCAAGACGGGGGGACTATATGGA
CTACTGGGGTCAAGGAACCTCAGTCACCGTCTCCTCAGCCAAGACCACCGCCCCC
AGCGTGTACCCCCTGGCCCCCGTGAGCAGCGGCAGCGGCAGCAGCAAGAACACA
CTTGTGCTCTTTGGGGCAGGATTCGGCGCAGTAATAACAGTCGTCGTCATCGTTG
TCATCATCAAATGCTTCTGTAAGCACAGAAGCTGTTTCAGAAGAAATGAGGCAA
GCAGAGAAACAAACAACAGCCTTACCTTCGGGCCTGAAGAAGCATTAGCTGAAC
AGACCGTCTTCCTTTAA
Sus scrofa (pig) 4-1BBL
SEQ ID NO: 6
ATGAGCTCCAGCTCCGACGTCGACCAGGACCCCGAGGCCCAGAGGCCTCCCGCG
CCCCGAGGCCGAGCTTGCAGCCCCTTGCCCTGGGCCTTGAGCGCCGCGCTGCTAG
TGTTCGCAGCCGCCAGCGCCGTCTGCCTGGTGCGCTCCTGGACCGCGCCCGGGGC
CCCCGCCGCGCCCGGTCCCGCGCCCAGCCCGCGACTGCCAGAGGGTCCGGAGTT
GACGTCCGACGCCCTGGCCGGCTTCCCAGACTATCCACAGGGCATGTTCCAGCAG
GATGTTTTCGCGCAGCTGGTGGTGGCGGAGGGTGAAAAGCTGACCGAAGGGCCC
CTGCACTGGTGCAGCGAGCCAGGGATGAAAGGTGTGACTCTGACGCCAGGTGTG
AGATATGACAGGGACACACATGAGCTGGTGGTGGTCAAGGCTGGAATCTACTAT
GTGTTCTTGCACTTGGCACTGAAGCGCGTGGTGGCCGTCAACAGCAACAGCAAC
AGCAACATCAGCTCCGGCTCCGTCTCCGTTGCCCTGCACCTGCAGCCTTCCCACG
CTGAGGCCACCGCCTTGGCCTTGACCTTGGACCTGCCACCCCCATCCTCAGGGAA
CTCAGCCGCTGGTTTCCGGGGTGGCCTGCTGCACCTGGGTGCTGGACAGCGCCTG
GGTGTCCACCTGCACCCTGAACTCCGCAAGCCCCTCACCTGGCAGCTCTTGGACA
CCACGGCCTTGGGCCTCTTCCGTGTGGCCACCAGAGCCCCTGCTGTACTCCCTTC
ACACCAGCTCTCATGA
Sus scrofa (pig) CD83 molecule
SEQ ID NO: 7
ATGGTGCTGGAGGTGAAAGTGGTCTGTTCGGAGGCTGTGGACTTGCCCTGCCCCG
TCCCCTGGGACCCGCAGGTCCGCTACACGGTGTCCTGGGCCAAGCTGTCCGGCGG
CCGTGCAGAGAGCCTGGAGGTGCCCGGGGAAGAGCTGCGGTCCGCTCTCCAGAA
TGGCTCTTCGGGGGCCCCCAGTGGGAGGCTGTATTCTCTGAAGATCCAGAACACT
ACCAGCTGCAACTCGGGGGCCTACCGCTGCACTCTGGAGGACCCGGAGACCCAG
AGAAACCTCAGCGGCACCGTGATCTTGAAAGTGACAGGATGCTCTAAGGGACGC
AAAGAAGATACTTTTAAGAAATACAGAGCTGAGATTGTCCTGCTGTTGGCTCTGG
TCACTTTCTACTTAACACTCATCATTTTCACCTGCAAGTTTGCACGACAGCAGAGT
ATTTTTCCAGATTTTTCTAAACCGGTCGTAGAACATGCTTTTCTCCCCGTCACCTC
CCCAAATAAGCCCTTGGAGCCAGTGACTCTTCACAAGACAGAGCTGGTA
Sus scrofa (pig) CD80 molecule
SEQ ID NO: 8
ATGTGTCACACACTGAAGTGGGGAACACCATTACCCAAGCTCTTTCAGCTCTTGG
TGCTGGTTGGTCTTTTTGACTTCTGTTCAGGCATCGTTCAGGTGACCAAAACAGTG
AAAGAAATAGCAGTGCTATCCTGTGATTACAACATATCCACTGAAGAACTGACT
AGAGTCCGAATATACTGGCAAAAGGATAATGAAATGGTGCTGGCTGTCATGTCT
GGAAAAGTGAAGGTGTGGCCCAAGTATGAGAACCGCACCTTCACTGATGTCACC
AATAACCTCTGCATTGTGATCCTGGCTCTGCGCCTGTCAGACAATGGCACCTACA
CCTGTGTTGTTCAGAAGCGGGAGAGAGGGTCTTATAAGCTGGAGCACCTGACTTC
GGTGAAGTTAATGGTCAAAGCTGACTTTCCTGTGCCTAGTATTACTGCCCTTGGA
AATCCATCTCCTAACATCAAAAGGATAAGGTGCTCAACCTCTGGAGGTTTTCCAG
AGCCTCACCTCTCCTGGTTGGAAAATGGAGAAGAATTAAATGCTACCAACACGA
TGCTTTCCCAAGATCCTGAAACTGAGCTCTACATGATTAGCAGTGAACTGGATTT
CAATGTGACAGGCAACCACAGCTTCATGTGTCTTGTCAAGTATGGAGGCTTAACA
GTGTCACAGACCTTCAACTGGCAAAAATCAGCTAAACGAGAAACCTCTTCTGCTA
ATCAGTTCCTGCTCTTCACCGTTATTATATCAGCCTCAGCATGTGGGATTGCAATA
ATACTAATATACCGGCTCTGCAGATCTGCTGCTCCAAGACAGAGAAGGAGGAGG
AATATGGAGAGCATGGAAATGGAAAGGATCTCCCCAATCTACCCAGGATCTGTG
GAAGGACCAGGCACC
Human FCGR1A or CD64
SEQ ID NO: 9
ATGTGGTTCTTGACAACTCTGCTCCTTTGGGTTCCAGTTGATGGGCAAGTGGACA
CCACAAAGGCAGTGATCACTTTGCAGCCTCCATGGGTCAGCGTGTTCCAAGAGG
AAACCGTAACCTTGCACTGTGAGGTGCTCCATCTGCCTGGGAGCAGCTCTACACA
GTGGTTTCTCAATGGCACAGCCACTCAGACCTCGACCCCCAGCTACAGAATCACC
TCTGCCAGTGTCAATGACAGTGGTGAATACAGGTGCCAGAGAGGTCTCTCAGGG
CGAAGTGACCCCATACAGCTGGAAATCCACAGAGGCTGGCTACTACTGCAGGTC
TCCAGCAGAGTCTTCACGGAAGGAGAACCTCTGGCCTTGAGGTGTCATGCGTGG
AAGGATAAGCTGGTGTACAATGTGCTTTACTATCGAAATGGCAAAGCCTTTAAGT
TTTTCCACTGGAATTCTAACCTCACCATTCTGAAAACCAACATAAGTCACAATGG
CACCTACCATTGCTCAGGCATGGGAAAGCATCGCTACACATCAGCAGGAATATC
TGTCACTGTGAAAGAGCTATTTCCAGCTCCAGTGCTGAATGCATCTGTGACATCC
CCACTCCTGGAGGGGAATCTGGTCACCCTGAGCTGTGAAACAAAGTTGCTCTTGC
AGAGGCCTGGTTTGCAGCTTTACTTCTCCTTCTACATGGGCAGCAAGACCCTGCG
AGGCAGGAACACATCCTCTGAATACCAAATACTAACTGCTAGAAGAGAAGACTC
TGGGTTATACTGGTGCGAGGCTGCCACAGAGGATGGAAATGTCCTTAAGCGCAG
CCCTGAGTTGGAGCTTCAAGTGCTTGGCCTCCAGTTACCAACTCCTGTCTGGTTTC
ATGTCCTTTTCTATCTGGCAGTGGGAATAATGTTTTTAGTGAACACTGTTCTCTGG
GTGACAATACGTAAAGAACTGAAAAGAAAGAAAAAGTGGGATTTAGAAATCTCT
TTGGATTCTGGTCATGAGAAGAAGGTAATTTCCAGCCTTCAAGAAGACAGACATT
TAGAAGAAGAGCTGAAATGTCAGGAACAAAAAGAAGAACAGCTGCAGGAAGGG
GTGCACCGGAAGGAGCCCCAGGGGGCCACGTGA
Human CD19scFv
SEQ ID NO: 10
ATGGCCTTACCAGTGACCGCCTTGCTCCTGCCGCTGGCCTTGCTGCTCCACGCCG
CCAGGCCGGACATCCAGATGACACAGACTACATCCTCCCTGTCTGCCTCTCTGGG
AGACAGAGTCACCATCAGTTGCAGGGCAAGTCAGGACATTAGTAAATATTTAAA
TTGGTATCAGCAGAAACCAGATGGAACTGTTAAACTCCTGATCTACCATACATCA
AGATTACACTCAGGAGTCCCATCAAGGTTCAGTGGCAGTGGGTCTGGAACAGAT
TATTCTCTCACCATTAGCAACCTGGAGCAAGAAGATATTGCCACTTACTTTTGCC
AACAGGGTAATACGCTTCCGTACACGTTCGGAGGGGGGACCAAGCTGGAGATCA
CAGGTGGCGGTGGCTCGGGCGGTGGTGGGTCGGGTGGCGGCGGATCTGAGGTGA
AACTGCAGGAGTCAGGACCTGGCCTGGTGGCGCCCTCACAGAGCCTGTCCGTCA
CATGCACTGTCTCAGGGGTCTCATTACCCGACTATGGTGTAAGCTGGATTCGCCA
GCCTCCACGAAAGGGTCTGGAGTGGCTGGGAGTAATATGGGGTAGTGAAACCAC
ATACTATAATTCAGCTCTCAAATCCAGACTGACCATCATCAAGGACAACTCCAAG
AGCCAAGTTTTCTTAAAAATGAACAGTCTGCAAACTGATGACACAGCCATTTACT
ACTGTGCCAAACATTATTACTACGGTGGTAGCTATGCTATGGACTACTGGGGCCA
AGGAACCTCAGTCACCGTCTCCTCA
Human optimized CD8α hinge and transmembrane domains:
SEQ ID NO: 11
ACTACGACTCCAGCACCACGACCACCAACACCAGCGCCCACCATTGCAAGCCAG
CCACTTTCACTGCGCCCAGAAGCGTGTAGACCAGCTGCTGGAGGAGCAGTGCAT
ACGAGAGGGCTGGACTTCGCCTGTGATATCTACATCTGGGCGCCCTTGGCCGGGA
CTTGTGGGGTCCTTCTCCTGTCACTGGTTATCACCCTTTACTGC
Porcine 4-1BB signaling domain
SEQ ID NO: 12
CAACAGGGCAGGAAGAAACTCCTGTATATAGTCAAACAACCGTTTCTGAAGCCA
GCGCAAACGGTCCAAGAGGAAGATGCCTGCAGCTGCCGGTTTCCAGAAGAAGAG
GAAGGCGAGTGTGAACTA
Porcine CD3 zeta signaling domain
SEQ ID NO: 13
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGGA
CAAGAGACGTGGCCGGGACCCTGAGATGGGGGGAAAGCCGAGGAGGAAGAACC
CTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAG
GCCCTGCCCCCTCGC
CD19scFv-hCD8-41BB-zeta CAR
SEQ ID NO: 14
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSSTTTPAPRPPTPAPTIASQPLSLRP
EACRPAAGGAVHTRGLDFACDIYIWAPLAGTCGVLLLSLVITLYCQQGRKKLLYIVK
QPFLKPAQTVQEEDACSCRFPEEEEGECELRVKFSRSADAPAYQQGQNQLYNELNLG
RREEYDVLDKRRGRDPEMGGKPRRKNPQEGLYNELQKDKMAEAYSEIGMKGERRR
GKGHDGLYQGLSTATKDTYDALHMQALPPR
Human CD19scFv
SEQ ID NO: 15
MALPVTALLLPLALLLHAARPDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLEITGGGGSGGGGSGGGGSEVKLQESGPGLVAPSQSLSVTCTVSGVSLP
DYGVSWIRQPPRKGLEWLGVIWGSETTYYNSALKSRLTIIKDNSKSQVFLKMNSLQT
DDTAIYYCAKHYYYGGSYAMDYWGQGTSVTVSS
Human optimized CD8α hinge and transmembrane domains
SEQ ID NO: 16
TTTPAPRPPTPAPTIASQPLSLRPEACRPAAGGAVHTRGLDFACDIYIWAPLAGTCGV
LLLSLVITLYC
Sus scrofa (pig) CD83 molecule
SEQ ID NO: 17
MSRGLQLLLLSCAYSLAPAEQKLISEEDLVLEVKVVCSEAVDLPCPVPWDPQVRYTV
SWAKLSGGRAESLEVPGEELRSALQNGSSGAPSGRLYSLKIQNTTSCNSGAYRCTLE
DPETQRNLSGTVILKVTGCSKGRKEDTFKKYRAEIVLLLALVTFYLTLIIFTCKFARQQ
SIFPDFSKPVVEHAFLPVTSPNKPLEPVTLHKTELV
Sus scrofa (pig) CD80 molecule
SEQ ID NO: 18
MCHTLKWGTPLPKLFQLLVLVGLFDFCSGIVQVTKTVKEIAVLSCDYNISTEELTRV
RIYWQKDNEMVLAVMSGKVKVWPKYENRTFTDVTNNLCIVILALRLSDNGTYTCV
VQKRERGSYKLEHLTSVKLMVKADFPVPSITALGNPSPNIKRIRCSTSGGFPEPHLSW
LENGEELNATNTMLSQDPETELYMISSELDFNVTGNHSFMCLVKYGGLTVSQTFNW
QKSAKRETSSANQFLLFTVIISASACGIAIILIYRLCRSAAPRQRRRRNMESMEMERISP
IYPGSVEGPGT
Human FCGR1A or CD64
SEQ ID NO: 19
MWFLTTLLLWVPVDGQVDTTKAVITLQPPWVSVFQEETVTLHCEVLHLPGSSSTQW
FLNGTATQTSTPSYRITSASVNDSGEYRCQRGLSGRSDPIQLEIHRGWLLLQVSSRVFT
EGEPLALRCHAWKDKLVYNVLYYRNGKAFKFFHWNSNLTILKTNISHNGTYHCSG
MGKHRYTSAGISVTVKELFPAPVLNASVTSPLLEGNLVTLSCETKLLLQRPGLQLYFS
FYMGSKTLRGRNTSSEYQILTARREDSGLYWCEAATEDGNVLKRSPELELQVLGLQL
PTPVWFHVLFYLAVGIMFLVNTVLWVTIRKELKRKKKWDLEISLDSGHEKKVISSLQ
EDRHLEEELKCQEQKEEQLQEGVHRKEPQGAT
pCD80-P2A-c-myc-pCD83
SEQ ID NO: 20
MCHTLKWGTPLPKLFQLLVLVGLFDFCSGIVQVTKTVKEIAVLSCDYNISTEELTRVR
IYWQKDNEMVLAVMSGKVKVWPKYENRTFTDVTNNLCIVILALRLSDNGTYTCVV
QKRERGSYKLEHLTSVKLMVKADFPVPSITALGNPSPNIKRIRCSTSGGFPEPHLSWL
ENGEELNATNTMLSQDPETELYMISSELDFNVTGNHSFMCLVKYGGLTVSQTFNWQ
KSAKRETSSANQFLLFTVIISASACGIAIILIYRLCRSAAPRQRRRRNMESMEMERISPI
YPGSVEGPGTVGSGATNFSLLKQAGDVEENPGPRSMATTMSRGLQLLLLSCAYSLAP
AEQKLISEEDLVLEVKVVCSEAVDLPCPVPWDPQVRYTVSWAKLSGGRAESLEVPG
EELRSALQNGSSGAPSGRLYSLKIQNTTSCNSGAYRCTLEDPETQRNLSGTVILKVTG
CSKGRKEDTFKKYRAEIVLLLALVTFYLTLIIFTCKFARQQSIFPDFSKPVVEHAFLPV
TSPNKPLEPVTLHKTELV
HA-pCD3scFv-mCD80TM
SEQ ID NO: 21
MRTPAQFLGILLLWFPGIKCYPYDVPDYAETTVTQSPASLSMAIGEQVTIRCITSTDID
DDMNWYQQRPGKPPKLLISEGNTLRPGVPSRFSSSGYGTDFVFTIENMLSEDVADYY
CLQSDNLPLTFGAGTKLELKGGGGSGGGGSGGGGSEVKLVESGGGLVKPGGSLKLS
CAASGFTFSSYTMSWVRQTPEKRLEWVAYISSGGSNSYYSDSVKGRFTISRDNARNT
LYLQMSSLRSEDTAMYYCARRGDYMDYWGQGTSVTVSSAKTTAPSVYPLAPVSSG
SGSSKNTLVLFGAGFGAVITVVVIVVIIKCFCKHRSCFRRNEASRETNNSLTFGPEEAL
AEQTVFL
hCD64-P2A-p41BBL-6xHis
SEQ ID NO: 22
MWFLTTLLLWVPVDGQVDTTKAVITLQPPWVSVFQEETVTLHCEVLHLPGSSSTQW
FLNGTATQTSTPSYRITSASVNDSGEYRCQRGLSGRSDPIQLEIHRGWLLLQVSSRVFT
EGEPLALRCHAWKDKLVYNVLYYRNGKAFKFFHWNSNLTILKTNISHNGTYHCSG
MGKHRYTSAGISVTVKELFPAPVLNASVTSPLLEGNLVTLSCETKLLLQRPGLQLYFS
FYMGSKTLRGRNTSSEYQILTARREDSGLYWCEAATEDGNVLKRSPELELQVLGLQL
PTPVWFHVLFYLAVGIMFLVNTVLWVTIRKELKRKKKWDLEISLDSGHEKKVISSLQ
EDRHLEEELKCQEQKEEQLQEGVHRKEPQGATVGSGATNFSLLKQAGDVEENPGPR
SMATTMSSSSDVDQDPEAQRPPAPRGRACSPLPWALSAALLVFAAASAVCLVRSWT
APGAPAAPGPAPSPRLPEGPELTSDALAGFPDYPQGMFQQDVFAQLVVAEGEKLTEG
PLHWCSEPGMKGVTLTPGVRYDRDTHELVVVKAGIYYVFLHLALKRVVAVNSNSN
SNISSGSVSVALHLQPSHAEATALALTLDLPPPSSGNSAAGFRGGLLHLGAGQRLGV
HLHPELRKPLTWQLLDTTALGLFRVATRAPAVLPSHQLSHHHHHH
Sus scrofa (pig) 4-1BBL (TNFSF9)
SEQ ID NO: 23
MSSSSDVDQDPEAQRPPAPRGRACSPLPWALSAALLVFAAASAVCLVRSWTAPGAP
AAPGPAPSPRLPEGPELTSDALAGFPDYPQGMFQQDVFAQLVVAEGEKLTEGPLHW
CSEPGMKGVTLTPGVRYDRDTHELVVVKAGIYYVFLHLALKRVVAVNSNSNSNISS
GSVSVALHLQPSHAEATALALTLDLPPPSSGNSAAGFRGGLLHLGAGQRLGVHLHPE
LRKPLTWQLLDTTALGLFRVATRAPAVLPSHQLS
Porcine 4-1BB signaling domain
SEQ ID NO: 24
QQGRKKLLYIVKQPFLKPAQTVQEEDACSCRFPEEEEGECEL
Porcine CD3 zeta signaling domain
SEQ ID NO: 25
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR

What is claimed is:

1. A genetically modified ungulate cell comprising a nucleic acid encoding a chimeric antigen receptor (CAR).

2. The genetically modified ungulate cell of claim 1, wherein the CAR comprises a human antigen binding domain operably linked to one or more ungulate intracellular signaling domains.

3. The genetically modified ungulate cell of claim 2, wherein the one or more ungulate intracellular signaling domains are porcine signaling domains.

4. The genetically modified ungulate cell of any one of claims 1-3, wherein the CAR is an anti-CD19 CAR, an anti-BCMA CAR, an anti-HER-2 CAR, an anti-EGFR CAR, or an anti-PSA CAR.

5. The genetically modified ungulate cell of claim 4, wherein the CAR is a single chain variable fragment (scFv).

6. The genetically modified ungulate cell of any one of claims 1-5, wherein the nucleic acid encoding the CAR comprises a nucleic acid sequence having at least 95% identity to SEQ ID NO: 1 or SEQ ID NO: 2.

7. The genetically modified ungulate cell of any one of claims 1-6, wherein the ungulate cell is selected from the group consisting of porcine, ovine and bovine cells.

8. The genetically modified ungulate cell of any one of claims 1-7, wherein the cell is selected from the group consisting of a monocyte, T lymphocyte, B lymphocyte, and natural killer (NK) cell.

9. A population of genetically modified ungulate cells comprising one or more genetically modified ungulate cells of any one of claims 1-8.

10. A pharmaceutical composition comprising:
(a) the genetically modified ungulate cell of any one of claims 1-8 or the population of claim 9; and a
(b) a pharmaceutically acceptable carrier.

11. A method for treating cancer in a subject comprising administering to the subject:
(a) the genetically modified ungulate cell of any one of claims 1-7;
(b) the population of genetically modified ungulate cells of claim 9; or
(c) the pharmaceutical composition of claim 10.

12. The method of claim 11, wherein the cancer comprises a solid tumor.

13. The method of claim 12, wherein the cancer is a hematological cancer.

14. The method of claim 12, wherein the cancer comprises a CD19-expressing cancer.

15. The method of any one of claims 11-14, further comprising administering a second anticancer therapy to the subject.

16. The method of claim 15, wherein the anticancer therapy is chemotherapy, immunotherapy, hormone therapy, cytokine therapy, radiotherapy, cryotherapy, or surgical therapy.

17. A method for activating ungulate T cells comprising:
(a) obtaining a population of ungulate T cells; and
(b) contacting the population of ungulate T cells with a population of artificial antigen presenting cells, wherein the antigen presenting cells express ungulate CD80, ungulate CD83, and an anti-ungulate CD3 antibody.

18. The method of claim 17, wherein the population of ungulate T cells is a population of porcine T cells and the anti-CD3 antibody is an anti-porcine CD3 antibody.

19. The method of claim 17 or 18, wherein the anti-CD3 antibody is a scFv anti-CD3 antibody.

20. The method of claim 18 or 19, wherein the population of porcine T cells are plated on a retronectin-coated dish in culture media comprising human IL-2 and porcine IL-21, prior to contacting the population of porcine T cells with the artificial antigen presenting cells.

21. The method of any one of claims 18-20, wherein contacting comprises stimulating the population of porcine T cells with the artificial antigen presenting cells at a ratio of 5:1 (porcine T cells: artificial antigen presenting cells).

22. A method for making a population of genetically modified ungulate T cells comprising a chimeric antigen receptor (CAR) comprising:
(a) obtaining a population of activated ungulate T cells produced by the method of any one of claims 17-21;
(b) introducing a nucleic acid encoding the CAR into the population of ungulate T cells.

23. The method of claim 22, wherein introducing the nucleic acid comprises transducing the population of activated ungulate T cells with a lentiviral vector comprising a nucleic acid construct encoding the CAR.

24. The method of claim 23, wherein the population of ungulate T cells is a population of porcine T cells.

25. The method of claim 24, wherein the population of porcine T cells is plated on retronectin-coated plates.

26. The method of claim 25, wherein the population of plated porcine T cells is transduced in the presence of polybrene.

27. The method of claim 26, further comprising expanding the population of plated transduced porcine T cells by culturing the population in culture media comprising human IL-2 and porcine IL-21.

28. The method of any one of claims 22-27, wherein a lentiviral vector comprises the nucleic acid encoding the CAR.

29. The method of any one of claims 22-28, wherein the CAR comprises a human antigen binding domain operably linked to one or more ungulate intracellular signaling domains.

30. The method of any one of claims 22-29, wherein the CAR is an anti-CD19 CAR, an anti-BCMA CAR, an anti-HER-2 CAR, an anti-EGFR CAR, or an anti-PSA CAR.

31. The method of any one of claims 22-30, wherein the CAR is a single chain variable fragment (scFv).

32. The method of any one of claims 24-31, herein the lentiviral vector comprises a construct and wherein the construct comprises
a. a nucleic acid sequence encoding an anti-CD19 scFv;
b. a nucleic acid encoding a CD8α hinge,
c. a CD8 transmembrane domain;
d. a porcine, cytoplasmic 41BB signaling domain; and
e. a cytoplasmic porcine CD3 zeta signaling domain, wherein the construct is operably linked to a promoter.

33. The method of claim 32, wherein the construct comprises a nucleic acid sequence having at least 95% identity to SEQ ID NO: 1 or SEQ ID NO: 2.

34. The method of claim 32 or 33, wherein the promoter is an EF1-α promoter.

35. The method of any of claims 22-33, further comprising introducing a nucleic acid encoding a heterologous polypeptide into the ungulate T cell or population of ungulate T cells.

36. The method of claim 35, wherein the heterologous polypeptide is selected from the group consisting of CD46, CD55 and CD59.

37. The method of any one of claims 22-36, further comprising incorporating the population of ungulate cells into a pharmaceutical composition.

38. The method of claim 37, wherein the ungulate cells are porcine cells.

39. A genetically modified ungulate cell comprising an oncolytic virus, wherein the cell is derived from a transgenic ungulate.

40. The genetically modified, ungulate cell of claim 39, wherein the transgenic ungulate cell is from a transgenic ungulate genetically modified to reduce rejection of a cell or tissue derived from the transgenic animal when transplanted into a human host.

41. The genetically modified, ungulate cell of claim 39 or 40, wherein the ungulate cell is selected from the group consisting of a porcine, ovine or bovine cell.

42. The genetically modified ungulate cell of claim 40 or 41, wherein the transgenic ungulate is genetically modified to reduce expression of alpha 1,3 galactosyltransferase; beta-2-microglobulin; T cell receptor alpha; T cell receptor beta;
or class II, major histocompatibility complex, transactivator (CIITA).

43. The genetically modified ungulate cell of claim 42, wherein the expression of alpha 1,3 galactosyltransferase; beta-2-microglobulin; T cell receptor alpha, T cell receptor beta, or class II, major histocompatibility complex, transactivator (CIITA) is conditionally reduced.

44. The genetically modified ungulate cell of any one of claims 40-43, wherein the transgenic ungulate is genetically modified to heterologously express a HLA-E-beta2M single chain trimer.

45. The genetically modified, ungulate cell of any one of claims 39-44, wherein the oncolytic virus is selected from the group consisting of vesicular stomatitis virus, measles, adenovirus, reovirus, herpes simplex virus, coxsackievirus A21, and vaccinia.

46. The genetically modified, ungulate cell of any one of claims 39-45, wherein the cell is selected from the group consisting of a monocyte, T lymphocyte, B lymphocyte, and natural killer (NK) cell.

47. The genetically modified, ungulate cell of any one of claims 39-46, wherein the cell is further genetically modified to enhance delivery of the oncolytic virus to a tumor cell.

48. The genetically modified, ungulate cell of claim 47, wherein the cell is genetically modified to express a receptor for the oncolytic virus.

49. The genetically modified, ungulate cell of claim 47, wherein the cell is genetically modified to disrupt expression of one or more proteins that prevent viral infection in an ungulate.

50. The genetically modified, ungulate cell of any one of claims 39-50, wherein the cell is genetically modified to express a chimeric antigen receptor (CAR).

51. The genetically modified, ungulate cell of claim 50, wherein the CAR comprises a human antigen binding domain operably linked to one or more porcine intracellular signaling domains.

52. The genetically modified, ungulate cell of claim 50 or 51, wherein the CAR is an anti-CD19 CAR, an anti-BCMA CAR, an anti-HER-2 CAR, an anti-EGFR CAR, or an anti-PSA CAR.

53. The genetically modified, ungulate cell of claim 52, wherein the CAR is a single chain variable fragment (scFv).

54. The genetically modified, ungulate cell of any one of claims 39-53, wherein the cell heterologously expresses CD46, CD55 or CD59.

55. A genetically modified ungulate cell comprising a chimeric antigen receptor (CAR), wherein the cell is derived from a transgenic ungulate.

56. The genetically modified, ungulate cell of claim 55, wherein the transgenic ungulate cell is from a transgenic ungulate genetically modified to reduce rejection of a cell or tissue derived from the transgenic animal when transplanted into a human host.

57. The genetically modified, ungulate cell of claim 55 or 56, wherein the ungulate cell is selected from the group consisting of a porcine, ovine or bovine cell.

58. The genetically modified ungulate cell of any one of claims 55-57, wherein the cell is derived from a transgenic ungulate, wherein the transgenic ungulate is:
(a) genetically modified to express a CAR; and
(b) genetically modified to reduce rejection of a cell or tissue derived from the transgenic animal when transplanted into a human host.

59. The genetically modified ungulate cell of claim 58, wherein the transgenic ungulate is genetically modified to reduce expression of alpha 1,3 galactosyltransferase; beta-2-microglobulin; one or more polypeptides of the T cell receptor complex; or class II, major histocompatibility complex, transactivator (CIITA).

60. The genetically modified ungulate cell of claim 59, wherein the expression of alpha 1,3 galactosyltransferase; beta-2-microglobulin; one or more polypeptides of the T cell receptor complex; or class II, major histocompatibility complex, transactivator (CIITA) is conditionally reduced.

61. The genetically modified ungulate cell of claim 59, wherein the transgenic ungulate is genetically modified to heterologously express a HLA-E-beta2M single chain trimer.

62. The genetically modified, ungulate cell of any one of claims 55-61, wherein the chimeric antigen receptor (CAR) comprises a human antigen binding domain operably linked to one or more porcine intracellular signaling domains.

63. The genetically modified, ungulate cell of claim 62, wherein the CAR is an anti-CD19 CAR, an anti-BCMA CAR, an anti-HER-2 CAR, an anti-EGFR CAR, or an anti-PSA CAR.

64. The genetically modified, ungulate cell of claim 63, wherein the CAR is a single chain variable fragment (scFv).

65. The genetically modified, ungulate cell of any one of claims 55-64, wherein the cell heterologously expresses C46, CD55 or CD59.

66. The genetically modified, ungulate cell of any one of claims 55-65, wherein the cell further comprises an oncolytic virus.

67. The genetically modified, ungulate cell of claim 66, wherein the oncolytic virus is selected from the group consisting of vesicular stomatitis virus, measles, coxsackievirus A21, adenovirus, reovirus, herpes simplex virus, and vaccinia.

68. The genetically modified, ungulate cell of any one of claims 55-67, wherein the cell is a T lymphocyte or a natural killer (NK) cells.

69. The genetically modified, ungulate cell of claim 68, wherein the cell is a T lymphocyte.

70. A population of genetically modified ungulate cells comprising one or more genetically modified ungulate cells of any one of claims 39-69.

71. A pharmaceutical composition comprising:
(a) the cell of any one of claims 39-69 or the population of claim 70; and
(b) a pharmaceutically acceptable carrier.

72. A transgenic ungulate comprising:
(a) two or more genetic modifications to reduce rejection of a cell or tissue derived from the transgenic ungulate when transplanted into a human host, wherein the modifications are selected from the group consisting of a modification that reduces expression of alpha 1,3 galactosyltransferase; a modification that reduces expression of beta-2-microglobulin; a modification that reduces expression of T cell receptor alpha; a modification that reduces expression of T cell receptor beta; a modification that reduces expression of class II, major histocompatibility complex; a modification that reduces expression of transactivator (CIITA); and an insertion of a nucleic acid sequence encoding a HLA-E-beta2M single chain trimer; and
(b) optionally comprising a nucleic acid sequence encoding a heterologous polypeptide.

73. The transgenic ungulate of claim 72, further comprising a nucleic acid sequence encoding a CAR.

74. The transgenic ungulate of claim 72 or 73, wherein the expression of alpha 1,3 galactosyltransferase; beta-2-microglobulin; T cell receptor alpha, T cell receptor beta, beta-2-microglobulin, or CIITA is conditionally reduced.

75. The transgenic ungulate of any one of claims 72-74, wherein the heterologous polypeptide is selected from the group consisting of CD46, CD55 and CD59.

76. The transgenic ungulate of any one of claims 72-75, wherein the transgenic ungulate is selected from the group consisting of a porcine, ovine or bovine animal.

77. A method for treating cancer comprising administering to a subject having cancer:
(a) the cell of any one of claims 39-69;
(b) the population of genetically modified ungulate cells of claim 70; or
(c) the pharmaceutical composition of claim 71.

78. The method of claim 77, wherein the cancer comprises a solid tumor.

79. The method of claim 77, wherein the cancer is a hematological cancer.

80. The method of claim 79, wherein the cancer comprises a CD19-expressing cancer.

81. The method of any one of claims 77-80, further comprising administering a second anticancer therapy to the subject.

82. The method of claim 81, wherein the anticancer therapy is chemotherapy, immunotherapy, hormone therapy, cytokine therapy, radiotherapy, cryotherapy, or surgical therapy.

83. A method for producing a genetically modified, ungulate cell as a carrier for an oncolytic virus, comprising:
(a) obtaining a cell or population of cells from a transgenic ungulate that is genetically modified to reduce rejection of a cell or tissue derived from the transgenic ungulate when transplanted into a human host and
(b) infecting the cell or population of cells with an oncolytic virus.

84. A method for producing a genetically modified, ungulate cell expressing a chimeric antigen receptor (CAR), comprising:
(a) introducing in vitro a nucleic acid encoding the CAR into a cell or population of cells derived from a transgenic ungulate genetically modified to reduce rejection of a cell or tissue derived from the transgenic animal when transplanted into a human host; or
(b) obtaining a cell or population of cells from a transgenic ungulate that is genetically modified to:
(i) express a CAR; and
(ii) genetically modified to reduce rejection of a cell or tissue derived from the transgenic ungulate when transplanted into a human host.

85. The method of claim 83 or 84, further comprising introducing a nucleic acid encoding a heterologous polypeptide into the cell or population of cells derived from the transgenic ungulate.

86. The method of claim 85, wherein the heterologous polypeptide is selected from the group consisting of CD46, CD55 and CD59.

87. The method of any one of claims 84-86, wherein the cell is selected from the group consisting of a T lymphocyte, a B lymphocyte, a natural killer (NK) cell, and a Monocyte.

88. The method of claim 87, further comprising expanding the population of genetically modified, ungulate cells.

89. The method of any one of claims 84-88, further comprising infecting the cell or population of cells with an oncolytic virus.

90. The method of any one of claims 84-89, further comprising incorporating the population of cells into a pharmaceutical composition.

91. The method of claim 90, wherein the oncolytic virus is selected from the group consisting of vesicular stomatitis virus, measles, adenovirus, reovirus, herpes simplex virus, coxsackievirus A21, and vaccinia.
